(19) United States
(12) Patent Application Publication
(10) Pub. No.: US 2019/0365675 A1
(43) Pub. Date: Dec. 5, 2019

(54) COMPOSITIONS FOR THE PREVENTION AND/OR TREATMENT OF ALCOHOL USE DISORDERS

(71) Applicants: SERVICIO ANDALUZ DE SALUD, Sevilla (ES); UNIVERSIDAD COMPLUTENSE DE MADRID, Madrid (ES)

(72) Inventors: Laura ORÍO ORTIZ, Madrid (ES); Fernando RODRÍGUEZ DE FONSECA, Sevilla (ES); María ANTÓN VALADÉS, Sevilla (ES)

(21) Appl. No.: 16/093,084

(22) PCT Filed: Apr. 11, 2017
(86) PCT No.: PCT/ES2017/070225

§ 371 Date: Aug. 19, 2019

(30) Foreign Application Priority Data
Apr. 11, 2016 (ES) P201630447

(51) Int. Cl. A61K 31/16; A61P 25/32; A23L 29/00

(52) U.S. Cl. CPC A61K 31/16; A61K 31/192; A23L 29/045; A61P 25/32

(57) ABSTRACT

The invention relates to the use of acylethanolamides in the production of a medicinal product or nutraceutical for the prevention, relief, and/or treatment of alcohol use disorders in general, and of alcohol intoxication or pathological inebriation, and alcohol dependence syndrome in particular.

### TECHNICAL FIELD OF THE INVENTION

The present invention is comprised in the field of medicine and pharmacy, and relates to the use of acylethanolamides in the production of a medicinal product or nutraceutical for the prevention, relief, and/or treatment of alcohol use disorders in general, and of alcohol intoxication or pathological inebriation, and alcohol dependence syndrome in particular.

### BACKGROUND OF THE INVENTION

The ICD-10 (acronym for the International Classification of Diseases, 10th revision) classifies under code F10 mental and behavioral disorders due to the use or consumption of alcohol.

Code F10 in turn comprises the following sub-sections:

F10.0. Acute intoxication.

F10.1. Harmful use.

F10.2. Dependence syndrome.

F10.3. Withdrawal syndrome.

F10.4. Withdrawal syndrome with delirium.

F10.5. Psychotic disorder.

F10.6. Amnesic syndrome.

F10.7. Residual and late-onset psychotic disorder.

F10.8. Other alcohol-induced mental disorders.

F10.9. Unspecified mental or behavioral disorder.

The clinical criteria for each diagnosis are included in the corresponding section in the chapter.

Harmful use (F10.1) is understood to mean that use which affects physical or mental health, but which does not meet the criteria of dependence or any other criteria indicated in F10.

All the most recent epidemiological studies on alcohol abuse and dependence conducted in Western countries confirm that harmful use is a growing and increasingly more serious issue, and in a particular manner, increasingly more among young people between 15 and 30 years old.

Most studies estimate that the likelihood of suffering alcohol dependence over a lifetime is currently about 9-10% for men and 3-5% for women; the same likelihood of practicing a harmful use would be virtually twice that.

Alcohol intoxication is a common pattern of alcohol consumption among some alcoholics or alcohol-dependent users. It has been proposed that alcohol-induced brain damage and neurodegeneration is a direct consequence of binging episodes, and there is convincing evidence which indicates that the neuroinflammation induced by these episodes may contribute to the neurotoxic effects of the drug.

A 4-day model of alcohol intoxication (12 intragastric doses), the so-called model of Majchrowicz intoxication, and minor modifications thereof, have been widely used to describe the neuroinflammation and neurodegenerative properties due to alcohol intoxication.

For example, alcohol intoxication causes microglia activation, increases DNA-binding activity of nuclear factor-Kappa B (NF kappa B), increases the expression of cyclooxygenase-2 (COX-2), and induces brain damage and neurodegeneration in the cerebral cortex and hippocampus associated with cognitive deficits. Furthermore, the mentioned model of alcohol intoxication induces prolonged high blood ethanol levels (BEL) similar to those documented in chronic alcoholics.

Chronic use of ethanol increases NF-kB-related inflammatory mediators and activates Toll-like receptor (TLR) 4 and 3 signaling pathways, inducing apoptosis, brain damage and neurodegeneration (Valles et al., 2004; Alfonso-Loeches et al., 2010; Qin & Crews, 2012). The neuroimmune activation induced by the chronic use of alcohol may involve the molecular alarm signal HMGB1 (High Mobility Group Box 1), which binds to TLR4 (Crews et al., 2013; Zou & Crews, 2014).

An increase in the post-mortem expression of HMGB1 in the orbitofrontal cortex of alcoholics has been observed and the levels thereof correlated with alcohol consumption while alive. Increases in peripheral proinflammatory cytokines, such as tumor necrosis factor alpha (TNF-α) and interleukin-1 beta (IL-1β), microglia activation, and an increase in the brain expression of monocyte chemoattractant protein 1 (MCP-1), have also been documented in alcohol-dependent humans. The inflammation also correlated with depressive symptoms and alcohol craving.

The endocannabinoid system has been studied for years because of its anti-inflammatory and homeostatic properties. A structural analogue of the endocannabinoid anandamide belonging to the N-acylethanolamine family, lipid mediator oleoylethanolamide (OEA), has emerged as an interesting bioactive molecule with anti-inflammatory and neuroprotective actions in the brain. OEA was first discovered as a satiety factor without activity at conventional cannabinoid receptors, and there is increasing evidence that it mediates a range of different actions through activation of peroxisome proliferator-activated receptor, alpha subtype (PPAR-α). Recent studies in animal models indicate that OEA may have putative neuroprotective properties against central nervous system (CNS) disorders such as apoplexy, Parkinson's disease, depression, or addiction.

Alcohol, specifically ethanol, is a potent psychoactive drug with a high number of side effects that may seriously affect the body. The mid- and long-term effects of alcohol vary and act on multiple organs and systems, as described in detail below.

In the brain and nervous system
Alcohol gradually affects brain functions, first affecting emotions (sudden mood swings), thought processes, and judgment. If the intake of alcohol continues, motor control is affected, causing dysarthria, dulling of the senses, and loss of balance.
It changes the action of neurotransmitters and modifies their structure and function. This causes a number of effects: reduced awareness, delayed reflexes, changes in vision, loss of muscle coordination, tremors, and hallucinations. It reduces self-control, and affects the memory, the ability to concentrate, and motor functions.
The drop in vitamin B1 caused by the chronic consumption of alcohol can lead to Wernicke-Korsakoff disease, which causes cognitive deficits and altered feelings, thoughts, and memory of a person. Those suffering this effect confuse reality with fantasy.
It causes sleep disorders.
Progressive loss of memory and other mental capabilities.
In advanced stages, it causes serious mental disturbances and irreversible brain damage, the so-called alcohol-related dementia.
Periods of amnesia, with a profound disturbance of the memory and the consciousness having a varying duration (minutes, hours, or even days).

In the heart and circulatory system.
It increases the cardiac activity (while a very moderate consumption improves circulation, a higher dose causes damage).
At high doses, alcohol increases blood pressure (hypertension) and causes damage to the heart muscle due to its toxic effects.
It weakens the heart musculature and, accordingly, the ability to pump blood.
It causes peripheral vasodilatation, which generates redness and an increase in the surface temperature of the skin.

In the digestive system.
Gastric discomforts are due to erosions in mucous membranes caused by ethanol. The heartburn will be more intense if different beverages have been mixed together or combined, because the gastric irritation is the result of all the components that are drank.
   Alcohol increases the production of gastric acid, which generates irritation and inflammation in the walls of the stomach so, in the long-term, ulcers, bleeding, and perforations of the gastric wall may occur.
   Stomach cancer has been related with alcohol abuse. It also causes cancer of the larynx, esophagus, pancreas, and bladder in some cases.
   It causes esophagitis, an inflammation of the esophagus, bleeding esophageal varices, and Mallory-Weiss tears.
   It can cause acute pancreatitis, a serious life-threatening inflammatory disease of the pancreas.
   It can cause chronic pancreatitis, which is characterized by permanent intense pain.
   Other possible disturbances are type 2 diabetes and peritonitis.
   The liver is the organ in charge of metabolizing alcohol, which is first transformed by liver enzymes into acetaldehyde and then into acetate and other compounds. This process is slow and is not damage-free (acetaldehyde depolarizes proteins, oxidizes lipids, consumes group B vitamins, and damages tissues).
   When the liver cell is irritated, it may cause alcoholic hepatitis due to cell destruction and tissue inflammation. Over time, the liver changes (fatty liver or steatosis) to adapt to the metabolic overload, and it may become hepatitis and latter cirrhosis of the liver, as a result of cell death and degeneration of the organ. This serious disease may ultimately degenerate into liver cancer and cause death.
   Other signs of liver disturbance are jaundice, a yellowish tone the skin and the sclera takes on, and edemas, an accumulation of fluid in the extremities.
   It disturbs renal function, reducing the levels of antidiuretic hormone, causing dehydration and taking water from other organs such as the brain, which causes a headache.
   Alcohol supplies abundant calories (7 kcal per gram of alcohol) with little nutritive value. It does not nourish, but it suppresses appetite, replaces other more complete foods, and in the long-term can cause malnourishment. This is worsened as it inhibits the absorption of some vitamins and minerals.

In blood.
Alcohol inhibits the production of white and red blood cells.
Without a sufficient amount of red blood cells to transport oxygen, megaloblastic anemia occurs.

In the immune and reproductive systems.
The lack of white blood cells leads to an immune system failure, increasing the risk of bacterial and viral infections.
It reduces the libido and sexual activity.
It can cause infertility and erectile dysfunction.

Furthermore, alcoholic patients usually present other associated psychiatric syndromes, particularly anxiety and depression, which are often disorders that are induced or aggravated by alcohol consumption itself.

Depression is a pathology that is often associated with alcoholism (36% of alcoholic patients suffer from depression at the same time), where said association is more common in women than in men, and it has a highly negative effect on the progression of alcoholic patients, increasing relapses of their disease and casting a shadow on the prognosis.

Medicinal products that are used today for treating alcoholism are usually aimed at treating the addiction, and sometimes at treating other associated diseases such as depression and anxiety. Table 1 shows the prevalence and Table 2 shows the active ingredients and the action pathway followed by the products that are used today for treating alcohol addiction.

TABLE 1
Worldwide prevalence (%) of alcohol addiction, 2013-2023.

| Data type | Age group | Intervention | 2013 | 2014 | 2015 | 2016 | 2017 | 2018 | 2019 | 2020 | 2021 | 2022 | 2023 |
|---|---|---|---|---|---|---|---|---|---|---|---|---|---|
| Prevalence (%) | > = 18 Years | Therapeutic | 2.79 | 2.79 | 2.79 | 2.80 | 2.80 | 2.80 | 2.81 | 2.81 | 2.81 | 2.81 | 2.82 |

TABLE 2
Products marketed for treating alcoholism.

| Name | Generic name | Target |
|---|---|---|
| piracetam | piracetam | Synaptic vesicle glycoprotein 2A (SV2A) |
| naltrexone ER | naltrexone | Mu-type opioid receptor (M-OR-1 or |
| | | MOR-1 or Mu opioid receptor or MOP |
| | | or OPRM1 or MOR1) |
| (bromazepam + sulpiride) | (bromazepam + sulpiride) | D(2) dopamine receptor (Dopamine D2 |
| | | receptor or DRD2 or D2DR); gamma- |
| | | aminobutyric acid (GABA) A receptor |
| | | (GABR) |
| (mecobalamin + piracetam) | (mecobalamin + piracetam) | Synaptic vesicle glycoprotein 2A (SV2A) |
| acamprosate calcium | acamprosate calcium | Gamma-aminobutyric acid (GABA) A |
| | | receptor (GABR) |
| alprazolam | alprazolam | Gamma-aminobutyric acid (GABA) A |
| | | receptor (GABR) |
| amitriptyline | amitriptyline [INN] | Sodium-dependent noradrenaline |
| | | transporter (Norepinephrine |
| | | transporter or NET or Solute Carrier |
| | | Family 6 Member 2 or SLC6A2); Sodium- |
| | | dependent serotonin transporter (5HT T |
| | | transporter or 5HTT or Solute Carrier |
| | | Family 6 Member 4 or SLC6A4) |
| calcium carbamide | calcium carbamide [INN] | Aldehyde Dehydrogenase(EC 1.2.1.3) |
| chlordiazepoxide | chlordiazepoxide hydrochloride | Gamma-aminobutyric acid (GABA) A |
| hydrochloride | | receptor (GABR) |
| clorazepate dipotassium | clorazepate dipotassium | Gamma-aminobutyric acid (GABA) A |
| | | receptor (GABR) |
| cyanamide | cyanamide | Aldehyde Dehydrogenase(EC 1.2.1.3) |
| diazepam | diazepam | Gamma-aminobutyric acid (GABA) A |
| | | receptor (GABR) |
| disulfiram | disulfiram [INN] | Aldehyde Dehydrogenase(EC 1.2.1.3) |
| fluoxetine hydrochloride | fluoxetine hydrochloride | Sodium-dependent serotonin |
| | | transporter (5HT T transporter or 5HTT |
| | | or Solute Carrier Family 6 Member 4 or |
| | | SLC6A4) |
| glutathione | | |
| haloperidol | haloperidol | D(2) dopamine receptor (Dopamine D2 |
| | | receptor or DRD2 or D2DR); |
| haloperidol decanoate | haloperidol decanoate | D(2) dopamine receptor (Dopamine D2 |
| | | receptor or DRD2 or D2DR); |
| hydroxyzine | hydroxyzine [INN] | Histamine H1 receptor (H1R or HRH1) |
| hydroxyzine hydrochloride | hydroxyzine hydrochloride | Histamine H1 receptor (H1R or HRH1) |
| imipramine | imipramine [INN] | Sodium-dependent noradrenaline |
| | | transporter (Norepinephrine |
| | | transporter or NET or Solute Carrier |
| | | Family 6 Member 2 or SLC6A2); Sodium- |
| | | dependent serotonin transporter (5HT T |
| | | transporter or 5HTT or Solute Carrier |
| | | Family 6 Member 4 or SLC6A4) |
| melperone | melperone [INN] | Dopamine receptor; 5- |
| | | Hydroxytryptamine (serotonin) receptor |
| meprobamate | meprobamate | Gamma-aminobutyric acid (GABA) A |
| | | receptor (GABR) |
| metadoxine | | |
| metronidazol | metronidazol | DNA |
| nalmefene | nalmefene | Delta-type opioid receptor (D-OR-1 or |
| | | DOR-1 or OPRD1 or OPRD); Mu-type |
| | | opioid receptor (M-OR-1 or MOR-1 or |
| | | mu opioid receptor or MOP or OPRM1 |
| | | or MOR1) |
| naltrexone hydrochloride | naltrexone hydrochloride | Mu-type opioid receptor (M-OR-1 or |
| | | MOR-1 or Mu opioid receptor or MOP |
| | | or OPRM1 or MORI) |
| nitrefazole | Nitrefazole [INN] | Aldehyde Dehydrogenase(EC 1.2.1.3) |
| piracetam | piracetam | Synaptic vesicle glycoprotein 2A (SV2A) |
| T.T.D B3 B4 | disulfiram + nicotinamide + | Aldehyde Dehydrogenase, |
| | adenine | Mitochondrial (ALDH Class 2 or ALDH-E2 |
| | | or ALDHI or EC 1.2.1.3); Dopamine Beta- |
| | | Hydroxylase (Dopamine Beta- |
| | | Monooxygenase or EC 1.14.17.1) |
| tiapride | tiapride [INN] | D(2) dopamine receptor (D2 receptor |
| | | and Dopamine or DRD2 or D2DR) |
| topiramate | topiramate | Gamma-aminobutyric acid (GABA) A |
| | | receptor (GABR); AMPA Receptor (GluR |
| | | or GRIA); Kainate receptor (KAR); |
| | | Voltage-gated sodium channel (SCN) |
| Ulcipep | chlordiazepoxide + clidinium | Muscarinic acetylcholine receptor M1 |
| | bromide | (CHRM1); Muscarinic acetylcholine |
| | | receptor M2 (CHRM2); Gamma- |
| | | aminobutyric acid (GABA) A receptor |
| | | (GABR) |
| acamprosate | acamprosate [INN] | Gamma-aminobutyric acid (GABA) A |
| | | receptor (GABR) |

For depression and alcohol addiction, (bromazepam+sulpiride), amitriptyline, fluoxetine hydrochloride are also being used.

For anxiety, (bromazepam+sulpiride), alprazolam, amitriptyline, chlordiazepoxide hydrochloride, clorazepate dipotassium, diazepam, hydroxyzine, hydroxyzine hydrochloride, meprobamate, tiapride, Ulcipep (chlordiazepoxide+clidinium bromide) are also indicated.

However, some of the abovementioned medicinal products have serious side effects, and the combined administration of several drugs is required for treatment of the symptoms and pathologies that accompany alcohol addiction. Therefore, it would be appropriate to develop treatment alternatives which had fewer side effects and which exerted their action on the highest possible number of the harmful effects of alcohol on the body, as well as preventive treatments for the damage caused by alcohol consumption at different levels and in different organs.

### BRIEF DESCRIPTION OF THE INVENTION

A first aspect of the present invention relates to the use of a compound of general formula (I) (also referred to as the compound of the invention):

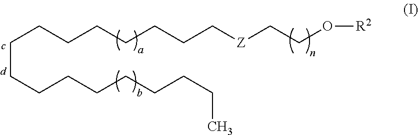

where
n is an integer between 0 and 5;
the sum of a and b can be from 0 to 4;
Z is a group which is selected from —C(O)N(R⁰)—; —(R⁰)NC(O)—; —(O)CO—; O; NR⁰; and S, and where R⁰ and R² are independently selected from the group consisting of a substituted or unsubstituted alkyl, hydrogen, a substituted or unsubstituted (C1-C6) alkyl, a substituted or unsubstituted (C1-C6) acyl, homoalkyl, and aryl, and where up to eight hydrogen atoms of the compound can be substituted by methyl or a double bond, and the molecular bridge existing between c and d can be saturated or unsaturated;
   or any of its pharmaceutically acceptable salts, esters, tautomers, solvates, and hydrates, or any of their combinations, in the production of a medicinal product or nutraceutical for the prevention, relief, improvement, and/or treatment of alcohol use or consumption disorders.

Alternatively, it relates to the compound of the invention or any of its salts, preferably any pharmaceutically acceptable salt, esters, tautomers, polymorphs, pharmaceutically acceptable hydrates, or an isomer, prodrugs, derivatives, solvates, or analogues, or any of their combinations, for use in the prevention, relief, improvement, and/or treatment of alcohol use or consumption disorders.

In another preferred embodiment of this aspect, the compound presents the formula (II):

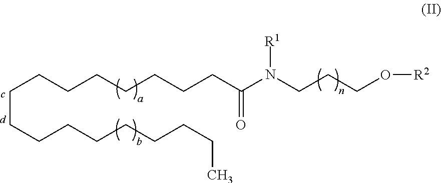

where
n is an integer between 0 and 4;
the sum of a and b can be from 1 to 3;
R¹ and R² are independent members which are selected from the group consisting of hydrogen (H), a substituted or unsubstituted (C1-C6) alkyl, a substituted or unsubstituted (C1-C6) acyl, and where up to eight hydrogen atoms of the compound can be substituted by methyl or a double bond, and the molecular bridge existing between c and d can be saturated or unsaturated;
   or any of its salts, preferably a pharmaceutically acceptable salt, esters, tautomers, polymorphs, pharmaceutically acceptable hydrates, or an isomer, prodrugs, derivatives, solvates, or analogues, or any of their combinations.

In another preferred embodiment of this aspect of the invention, a=1 and b=1.

In another preferred embodiment of this aspect of the invention, n=0-1.

In another preferred embodiment of this aspect of the invention, R¹ and R² are hydrogens (H).

In another preferred embodiment of this aspect of the invention, the bridge between carbon c and carbon d is a double bond.

In another preferred embodiment of this aspect of the invention, the compound of formula (I) is an acylethanolamide. More preferably, the acylethanolamide is selected from the list consisting of oleoylethanolamide (OEA), palmitoylethanolamide (PEA), stearoylethanolamide (SEA), or any of their combinations. Even more preferably, the acylethanolamide is oleoylethanolamide (OEA) of formula (III).

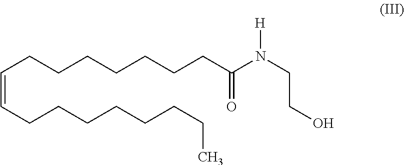 [SMILES: CCCCCCCC/C=C\CCCCCCCC(=O)NCCO]

In another preferred embodiment of the first aspect of the invention, the alcohol use or consumption disorders are selected from alcohol intoxication or pathological inebriation, and alcohol dependence syndrome.

In another more preferred embodiment, the alcohol consumption disorder is alcohol intoxication or pathological inebriation.

In another even more preferred embodiment, the alcohol consumption disorders (or symptoms) are selected from the list consisting of neuroinflammation, neurotoxicity, neuronal death, liver damage, veisalgia, or any of their combinations.

In another more preferred embodiment, the alcohol consumption disorder is alcohol dependence syndrome. In another even more preferred embodiment, the alcohol use or consumption disorders (or symptoms) are selected from the list consisting of neuroinflammation, neurotoxicity, neuronal death, liver damage, veisalgia, anhedonia, compulsion for, tolerance to, and inability to control alcohol consumption, withdrawal comprising anxiety and depression, or any of their combinations.

In another more preferred embodiment, the alcohol use or consumption disorder (or symptom) associated with alcohol dependence syndrome is neuroinflammation, neurotoxicity, neuronal death, veisalgia, and/or liver damage, or any of their combinations.

In another more preferred embodiment, the alcohol use or consumption disorder (or symptom) associated with alcohol dependence syndrome is anhedonia.

In another more preferred embodiment, the alcohol use or consumption disorder (or symptom) associated with alcohol dependence syndrome is the compulsion for, tolerance to, and/or inability to control alcohol consumption.

In another more preferred embodiment, the alcohol use or consumption disorder (or symptom) associated with alcohol dependence syndrome is anxiety.

In another more preferred embodiment, the alcohol use or consumption disorder (or symptom) associated with alcohol dependence syndrome is depression.

In another preferred embodiment of the first aspect of the invention, the use is preventive and the administration of the compound of the invention is performed before the intake of alcohol.

In another preferred embodiment of this aspect, the use is preventive and the administration of the compound of the invention is performed during the intake of alcohol.

A second aspect of the invention, relates to the use of a pharmaceutical composition comprising or consisting of at least one of the compounds of formula (I), formula (II), and/or formula (III) as defined in the present invention, or any of their pharmaceutically acceptable salts, esters, tautomers, solvates, and hydrates, or any of their combinations, in the production of a medicinal product for the prevention, relief, improvement, and/or treatment of alcohol use or consumption disorders.

Alternatively, the second aspect of the invention relates to a pharmaceutical composition comprising or consisting of at least one of the compounds of formula (I), formula (II), and/or formula (III) as defined in the present invention, or any of their pharmaceutically acceptable salts, esters, tautomers, solvates, and hydrates, or any of their combinations, for use in the prevention, relief, improvement, and/or treatment of alcohol use or consumption disorders.

In a more preferred embodiment, the composition further comprises or consists of one or more pharmaceutically acceptable excipients. In an even more preferred embodiment, the composition further comprises or consists of another active ingredient. Preferably the active ingredient is selected from the list comprising or consisting of ibuprofen, paracetamol, vitamin B12, caffeine, or any of their combinations.

In another preferred embodiment of this second aspect of the invention, the alcohol use disorders are selected from alcohol intoxication or pathological inebriation, and alcohol dependence syndrome.

A third aspect of the invention relates to a food composition, a nutraceutical composition, or a “medical food” type composition, hereinafter food composition of the invention, comprising or consisting of at least one of the compounds of formula (I), formula (II), and/or formula (III) as defined in the present invention, or any of their combinations,

A fourth aspect of the invention relates to the use of the food composition of the invention for the prevention, relief, improvement, and/or treatment of alcoholism or of a disease or pathological condition caused by the intake of alcohol in a mammal. Alternatively, it relates to the use of the food composition of the invention for the prevention and/or treatment of alcoholism or of a disease or pathological condition caused by the intake of alcohol in a mammal.

In another preferred embodiment, it relates to the use of a food or “medical food” type composition comprising or consisting of at least one of the compounds of formula (I), formula (II), and/or formula (III) as defined in the present invention, or any of their combinations, in the production of a nutraceutical for the improvement of physiological functions, for the prevention, treatment, or improvement of the quality of life in alcohol use disorders.

In a preferred embodiment, the composition further comprises another food. In an even more preferred embodiment, the food is selected from the list comprising or consisting of preparations based on electrolytes and/or isotonic beverages, natural juices, thistle teas, caffeine preparations, or any of their combinations.

In a preferred embodiment of this aspect of the invention, the alcohol use disorders are selected from alcohol intoxication or pathological inebriation, and alcohol dependence syndrome. In a more preferred embodiment, the disorders or symptoms of alcohol intoxication or pathological inebriation are selected from the list consisting of neuroinflammation, neurotoxicity, neuronal death, liver damage, and/or veisalgia, or any of their combinations. In another more preferred embodiment, the disorders or symptoms of alcohol dependence syndrome are selected from the list consisting of neuroinflammation, neurotoxicity, neuronal death, veisalgia, liver damage, anhedonia, compulsion for, tolerance to, and inability to control alcohol consumption, withdrawal, especially anxiety and depression, or any of their combinations.

### DESCRIPTION OF THE FIGURES

FIG. 1. Experimental design of the study.

A) Experimental protocol of excessive alcohol consumption. The rats received an initial dose of 5 g/kg of 30% ethanol (w/v) through an oral gavage and a maximum of 3 g/kg of ethanol in subsequent doses every 8 h. Blood was taken from the tail vein 2 h before and 2 h after the 15:00 h administration of ethanol by a gavage on days 2 to 4 for the purpose of determining blood ethanol levels (BEL), and the attempt was made to keep the toxic doses of ethanol BEL relatively constant (see Table 3). The mean doses of ethanol/rat were 8 g/kg, 7.5 g/kg, 7.9 g/kg and 4.9 g/kg, on days 1 to 4, respectively, and the mean dose of ethanol/rat/day (1-4) was 7.06 g/kg. In a first experiment, the time-course of neuroinflammation was checked after 1 h, 6 h, and 24 h after the last ethanol force-feeding. In the second experiment, OEA (5 mg/kg, i.p., loading dose of 10 mg/kg) was injected as a pre-treatment 10 min before each ethanol force-feeding, and the brain tissue/blood samples were collected 2-4 h after the last administration of ethanol.

B) Proposed TLR4/MyD88/NF-kB neuroimmune signaling pathway mediated by HMGB1 after alcohol-induced activation, causing oxidative stress, caspase 3 activation, and cell damage. HMGB1 is a dangerous cytokine that is recruited by alcohol and activates the MyD88-dependent TLR4 immune signaling pathway, which induces translocation of the p65 subunit of NF-kB to the nucleus and increases its transcriptional activity. The proinflammatory cascade mediated by NF-kB involves the release of cytokines, such as TNF-α and IL-1β, inducing greater activation of NF-kB and more neuroinflammation, and chemokine MCP-1 (which is also induced by a change in the oxidative stress), which mediates recruitment in the microglia and increases neurodegeneration. The transcriptional activity of NF-kB induces the expression of other proinflammatory markers, such as COX-2 and iNOS, which leads to oxidative and nitrosative stress. Lipid peroxidation, measured by the accumulation of 4-HNE, induces a redox cell state related to caspase 3 activation and apoptotic cell death mediated by caspase 8.

C) Experimental design for behavioral studies during the acute ethanol withdrawal period. The animals were tested in the elevated plus maze 24 hours and 12 days after the alcohol abuse protocol and the forced swim test was performed 48 hours after the last administration of ethanol. [HMGB1: (High Mobility Group Box 1); TLR4 (Toll-Like Receptors 4); MD2 (Myeloid Differentiation Protein 2); MyD88 (Myeloid Differentiation Factor 88); NF-kB (Nuclear Factor kB) (p65 subunit); IkB: IkappaB inhibitor protein; TNF-α (Tumor Necrosis Factor α); IL-1β (Interleukin 1β); MCP-1 (Monocyte Chemoattractant Protein 1); iNOS (inducible Nitric Oxide Synthase); COX-2 (Cyclooxygenase 2); 4-HNE (4-hydroxynonenal)].

FIG. 2. Time-course of the increase in proinflammatory mediators in the frontal cortex and of blood corticosterone induced by excessive ethanol consumption. The parameters were measured 1 h, 6 h, and 24 h after the last administration of ethanol. A) TNF-α levels were over-expressed in the frontal cortex 6 h after treatment with excess ethanol and were under-expressed 24 h after treatment; B) IL-1β levels showed a tendency to increase after treatment with excess ethanol, but were not significantly high; C) The activity of p65 subunit of NFkB increased and decreased in the nuclear extracts of the frontal cortex 6 h and 24 h after excess ethanol, respectively; D) IkBα levels were high at all times after treatment with excess ethanol; E) Enzyme COX-2 was over-expressed 6 h after exposure to ethanol; F) Treatment with excess ethanol increases blood corticosterone levels 1 h, 6 h, and 24 h after the last ethanol force-feeding. The data represents the mean±S.E.M. (n=3-6). Different from the control group: *p<0.05; **p<0.01; ***p<0.001.

FIG. 3. Effects of the pre-treatment with OEA on the HMGB1/TLR4 signaling pathway activated in the frontal cortex after exposure to excessive ethanol consumption. A) HMGB1 levels measured by ELISA (enzyme-linked immunosorbent assay); B) Nuclear/cytoplasmic ratio of HMGB1 analyzed by means of Western blot; the data is expressed as a percentage of the controls in each fraction. The expression of HMGB1 in the control animals was higher in the nucleus than in the cytoplasm (n/c ratio=1.3); C) Relative TLR4 mRNA levels; D) TLR4 protein levels; E) MD2 protein levels; F) MyD88 protein levels. Parameters measured 2-4 h after the last administration of ethanol. The data represents the mean±S.E.M. (n=4-10). mRNA levels were normalized by means of GAPDH and densitometric data in Western blot analysis, the bands of interest were normalized by ß-actin (lower band). Different from the control group: *p<0.05; **p<0.01; ***p<0.001. Different from the animals treated with ethanol: #p<0.05; ##p<0.01.

FIG. 4. Release of TNF-α, IL-1β and MCP-1 in the frontal cortex and/or plasma after the pharmacological treatments. ELISA (enzyme-linked immunosorbent assay) data of the levels in the frontal cortex (left panel) of TNF-α (A), IL-β (C) and MCP-1 (E). ELISA (enzyme-linked immunosorbent assay) detected plasma levels (right panel) of TNF-α (B) and IL-β (D). Parameters measured 2-4 h after the last administration of ethanol. The data represents the mean±S.E.M. (n=3-6). Different from the control group: *p<0.05; **p<0.01; ***p<0.001. Different from the animals treated with ethanol: #p<0.05; ##p<0.01; ###p<0.001.

FIG. 5. Activation of NFkB in the frontal cortex induced by exposure to excessive ethanol consumption and effects of the pre-treatment with OEA. A) Relative mRNA levels of the nuclear subunit p65 of NFkB; B) mRNA levels of cytosol NFkB inhibitory protein IκBα; C) Nuclear activity of p65 measured by a kit based on ELISA (enzyme-linked immunosorbent assay); D) Protein levels of IκBα in cytosol extracts. Parameters measured 2-4 h after the last administration of ethanol. The data represents the mean±S.E.M. (n=4-10). The densitometric data of IκBα was normalized by ß-actin (lower band). Different from the control group: *p<0.05; **p<0.01; ***p<0.001. Different from the animals treated with ethanol: #p<0.05; ##p<0.01.

FIG. 6. Role of OEA in the overexpression of the regulation of iNOS and COX-2, lipid peroxidation, caspase 8 and caspase 3 induced by ethanol and activity in the frontal cortex. A) Relative iNOS mRNA levels; B) Relative COX-2 mRNA levels; C) Relative 4-hydroxynonenal (4-HNE) levels, a natural product of lipid peroxidation; D) Relative caspase 8 mRNA levels, a signaling intermediary of caspase 3 activation; E) caspase 3 protein levels; F) caspase 3 activity measured by fluorometric assay. Parameters measured 2-4 h after the last administration of ethanol. The data represents the mean±S.E.M. (n=4-10). Different from the control group: *p<0.05; **p<0.01; ***p<0.001. Different from the animals treated with ethanol: #p <0.05; ## p<0.01.

FIG. 7. Role of OEA in the overexpression of plasma corticosterone levels induced by ethanol and the intestinal permeability to LPS. A) Plasma corticosterone levels detected by radioimmunoassay (R.I.A.); B) Plasma LPS levels detected by enzymatic analysis. Parameters measured 2-4 h after the last administration of ethanol. The data represents the mean±S.E.M. (n=6-9). Different from the group of control: *p<0.05; **p<0.01. Different from the animals treated with ethanol: #p<0.05.

FIG. 8. Anxiolytic and anti-depressant type effects of OEA in state of acute withdrawal for excessive ethanol consumption. OEA was administered as pre-treatment before each ethanol force-feeding and the animals were subjected to the forced swim test (upper panel) and the elevated plus maze (lower panel) 48 h and 24 h/12 days, respectively, after the alcohol excess protocol. In the forced swim test, drops in the times of swimming (A), climbing (B) and latency (D) and the increase in immobility (C) are indexes of depressive-like behavior. In the elevated plus maze, a drop in the percentage of entries in the open arms (E, G) and in the percentage of time used in the open arms (F, H) are considered anxiety measurements. The data represents the mean±S.E.M. (n=5-8). Different from the control group: *p<0.05; **p<0.01. Different from the animals treated with ethanol: #p<0.05.

### DETAILED DESCRIPTION OF THE INVENTION

The authors of the present invention have characterized the temporal profile of neuroinflammation in rats exposed to the excessive intragastric administration of ethanol (3 times/day×4 days), and the anti-inflammatory/neuroprotective properties of oleoylethanolamide (OEA) were assayed.

The authors of the present invention have also seen that OEA, administered in pre-treatment during alcohol intoxication, exerts antidepressant-like effects during acute withdrawal. As a whole, the results clearly show a beneficial profile of OEA as a potent anti-inflammatory, antioxidant, neuroprotective, and antidepressant compound for treating alcohol intoxication and the disorders associated with alcohol use or consumption.

It should be noted that the results described in detail in the present invention may be generalized to fatty acid ethanolamides, therefore, the invention relates to the use of a fatty acid ethanolamide in the production of a medicinal product for the prevention, relief, and/or treatment of a disease caused by the intake of alcohol, and preferably, a hangover.

### Medical Use of the Compound of the Invention

Therefore, a first aspect of the present invention relates to the use of a compound of general formula (I) (also referred to as the compound of the invention):

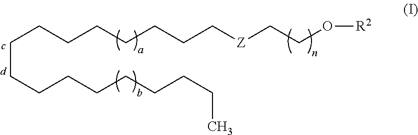

where
n is an integer between 0 and 5,
the sum of a and b can be from 0 to 4, i.e. 0≤(a+b)≥4,
Z is a group which is selected from —C(O)N(R⁰)—; —(R⁰)NC(O)—; —(O)CO—; O; NR⁰; and S, and where R⁰ and R² are independently selected from the group consisting of a substituted or unsubstituted alkyl, hydrogen, a substituted or unsubstituted (C1-C6) alkyl, a substituted or unsubstituted (C1-C6) acyl, homoalkyl, and aryl, and where up to eight hydrogen atoms of the compound can be substituted by methyl or a double bond, and the molecular bridge existing between c and d can be saturated or unsaturated;
   or any of its pharmaceutically acceptable salts, esters, tautomers, solvates, and hydrates, a derivative or a prodrug thereof, or any of their combinations, in the production of a medicinal product or a nutraceutical for the prevention, relief, and/or treatment of alcohol use or consumption disorders in a mammal.

Alternatively, it relates to the compound of the invention or any of its salts, preferably any pharmaceutically acceptable salt, esters, tautomers, polymorphs, pharmaceutically acceptable hydrates, or an isomer, prodrugs, derivatives, solvates, or analogues, a derivative or a prodrug thereof, or any of their combinations, for use in the prevention, relief, and/or treatment of alcohol use or consumption disorders in a mammal.

In another preferred embodiment of this aspect, the compound of the invention presents the formula (II):

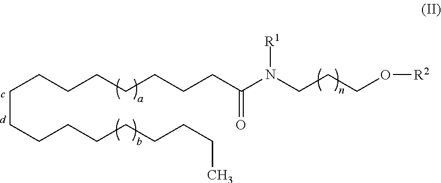

where
n is a number between 0 and 4;
the sum of a and b can be from 1 to 3, i.e. 0≤(a+b)≥3;
R1 and R2 are independent members which are selected from the group consisting of hydrogen (H), a substituted or unsubstituted (C1-C6) alkyl, a substituted or unsubstituted (C1-C6) acyl, and where up to eight hydrogen atoms of the compound can be substituted by methyl or a double bond, and the molecular bridge existing between c and d can be saturated or unsaturated;
   or any of its salts, preferably a pharmaceutically acceptable salt, esters, tautomers, polymorphs, pharmaceutically acceptable hydrates, or an isomer, prodrugs, derivatives, solvates, or analogues, or any of their combinations.

In another preferred embodiment of this aspect of the invention, a=1 and b=1.

In another preferred embodiment of this aspect of the invention, n=0-1.

In another preferred embodiment of this aspect of the invention, R¹ and R² are hydrogens (H).

In another preferred embodiment of this aspect of the invention, the bridge between carbon c and carbon d is a double bond.

In another preferred embodiment of this aspect of the invention, the compound of formula (I) is an acylethanolamide. More preferably, the acylethanolamide is selected from the list consisting of oleoylethanolamide (OEA), palmitoylethanolamide (PEA), stearoylethanolamide (SEA), or any of their combinations. Even more preferably, the acylethanolamide is the oleoylethanolamide (OEA) of formula (III).

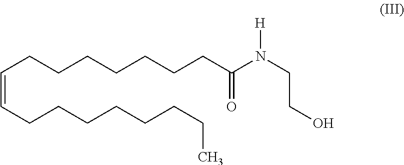 [SMILES: CCCCCCCC/C=C\CCCCCCCC(=O)NCCO]

The term “palmitoylethanolamide” refers to the compound the structure of which is

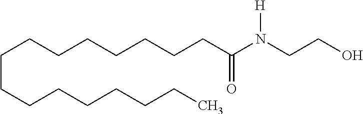 [SMILES: CCCCCCCCCCCCCCCCC(=O)NCCO]

In other aspects, the invention relates to fatty acid ethanolamide compounds, homologues, analogues; and their pharmaceutical compositions, as well as to uses thereof.

In other embodiments, the fatty acid moiety of the fatty acid alkanolamide or ethanolamide compound, homologue or analogue, can be saturated or unsaturated, and if unsaturated it can be monounsaturated or polyunsaturated.

In some embodiments, the fatty acid moiety of the fatty acid alkanolamide compound, homologue or analogue, is a fatty acid selected from the group consisting of oleic acid, palmitic acid, elaidic acid, palmitoleic acid, linoleic acid, α-linolenic acid, and γ-linolenic acid. In certain embodiments, the fatty acid moieties have from 12 to 20 carbon atoms.

Other embodiments relate to compounds which are obtained by means of varying the hydroxyalkylamide fraction of the fatty acid amide compound, its homologue, or its analogue. These embodiments include the introduction of a substituted or unsubstituted alkyl group of one to three carbon atoms (C1-C3) in the hydroxyl group of an alkanolamide or ethanolamide moiety for the purpose of forming the corresponding lower alkyl ether. In another embodiment, the hydroxy group of the alkanolamide or ethanolamide moiety binds to a carboxylate group of a substituted or unsubstituted alkyl group of 2 to 6 atoms (C₂ to C₆) of alkyl carboxylic acid to form the corresponding ester of the fatty acid ethanolamide. Such embodiments include fatty acid alkanolamide and fatty acid ethanolamides in the ester bond of organic carboxylic acids such as acetic acid, propionic acid, and butanoic acid. In another embodiment, the fatty alkanolamide acid is oleoylalkanolamide. In a much more preferred additional embodiment, the fatty acid alkanolamide is oleoylethanolamide.

In another embodiment of this aspect of the invention, the fatty acid ethanolamide compound, homologue, or analogue further comprises a substituted or unsubstituted alkyl group (—C3) covalently bound to the nitrogen atom of the fatty acid ethanolamide.

In the present invention, the term “alkyl” refers to radicals of linear or branched hydrocarbon chains, having 1 to 10 carbon atoms, preferably 1 to 4, and they are bound to the moiety of the molecule by means of a single bond, for example, methyl, ethyl, n-propyl, i-propyl, n-butyl, tert-butyl, sec-butyl, n-pentyl, n-hexyl, etc. The alkyl groups can optionally be substituted with one or more substituents such as halogen, hydroxyl, alkoxyl, carboxyl, carbonyl, cyano, acyl, alkoxycarbonyl, amino, nitro, mercapto, and alkylthio.

The term “alkenyl” refers to radicals of hydrocarbon chains containing one or more carbon-carbon double bonds, for example, vinyl, 1-propenyl, allyl, isoprenyl, 2-butenyl, 1,3-butadienyl, etc. The alkenyl radicals can optionally be substituted with one or more substituents such as halo, hydroxyl, alkoxyl, carboxyl, cyano, carbonyl, acyl, alkoxycarbonyl, amino, nitro, mercapto and alkylthio.

The compounds of the present invention represented by formula (I), (II), or (III) may include isomers, depending on the presence of multiple bonds, including optical isomers or enantiomers, depending on the presence of chiral centers. The individual isomers, enantiomers, or diastereoisomers and the mixtures thereof fall within the scope of the present invention, i.e., the term isomer also refers to any mixture of isomers, such as diastereomers, racemates, etc., including the optically active isomers thereof or the mixtures in different proportions thereof. The individual enantiomers or diastereoisomers, as well as their mixtures, can be separated by means of conventional techniques.

Likewise, included within the scope of this invention are the prodrugs of the compounds of formula (I). As it is used herein, the term “prodrug” includes any derivative of a compound of formula (I), as a non-limiting example, esters (including carboxylic acid esters, amino acid esters, phosphate esters, metal salt sulfonate esters, etc.), carbamates, amides, etc., which, when administered to an individual, can be transformed directly or indirectly into said compound of formula (I) in the mentioned individual. Advantageously, said derivative is a compound which increases the bioavailability of the compound of formula (I) when it is administered to an individual, or enhances the release of the compound of formula (I) in a biological compartment. The nature of said derivative is not critical provided that it can be administered to an individual and provides the compound of formula (I) in a biological compartment of an individual. The preparation of said prodrug can be carried out by means of conventional methods known by those skilled in the art.

As it is used herein, the term “derivative” includes both pharmaceutically acceptable compounds, i.e., derivatives of the compound of formula (I) which can be used in the production of a medicinal product or food compositions, and pharmaceutically unacceptable derivatives, since these can be useful in the preparation of pharmaceutically acceptable derivatives.

The compounds of the invention can be in crystalline form as free compounds or as solvates. In this sense, as it is used herein, the term “solvate” includes both pharmaceutically acceptable solvates, i.e., solvates of the compound of formula (I) which can be used in the production of a medicinal product, and pharmaceutically unacceptable solvates, which can be useful in the preparation of pharmaceutically acceptable solvates or salts. The nature of the pharmaceutically acceptable solvate is not critical provided that it is pharmaceutically acceptable. In a particular embodiment, the solvate is a hydrate. The solvates can be obtained by conventional solvation methods known by those skilled in the art.

For their application in therapy, the compounds of formula (I), their salts, prodrugs, or solvates, will preferably be in a pharmaceutically acceptable or substantially pure form, i.e., they have a pharmaceutically acceptable level of purity excluding the usual pharmaceutical additives such as diluents and carriers, and not including material considered toxic a normal dosage levels. The levels of purity for the active ingredient are preferably greater than 50%, more preferably greater than 70%, and still more preferably greater than 90%. In a preferred embodiment, they are greater than 95% of compound of formula (I), or of its salts, solvates, or prodrugs.

In another preferred embodiment of the first aspect of the invention, the alcohol use or consumption disorders are selected from alcohol intoxication or pathological inebriation, and alcohol dependence syndrome.

In a more preferred embodiment, the alcohol use disorder is alcohol intoxication or pathological inebriation.

The examples of the present invention show evidence of the anti-inflammatory and neuroprotective effects induced by OEA. The results indicate that OEA interferes with the HMGB1/TLR4/MyD88 neuroimmune danger signal associated with the proinflammatory cascade mediated by NF-κB and protects against hyperactivity of proapoptotic caspase-3 enzyme in rat frontal cortex, all caused by ethanol intoxication. Furthermore, OEA inhibited activation of the hypothalamic-pituitary-adrenal (HPA) axis under exposure to alcohol intoxication without disturbing ethanol metabolism.

In an even more preferred embodiment, the disorders or symptoms are selected from the list consisting of neuroinflammation, neurotoxicity, neuronal death, liver damage, veisalgia, or any of their combinations.

In a more preferred embodiment, the alcohol use or consumption disorder is alcohol dependence syndrome. In an even more preferred embodiment, the disorders or symptoms are selected from the list consisting of neuroinflammation, neurotoxicity, neuronal death, liver damage, veisalgia, anhedonia, compulsion for, tolerance to, and inability to control alcohol consumption, withdrawal comprising anxiety and depression, or any of their combinations.

More preferably, the disorder or symptom associated with alcohol dependence syndrome is neuroinflammation, neurotoxicity, neuronal death, veisalgia, and/or liver damage, or any of their combinations.

Even more preferably, the disorder or symptom due to alcohol use or consumption is hangover or veisalgia.

More preferably, the disorder or symptom associated with alcohol dependence syndrome is anhedonia.

More preferably, the disorder or symptom associated with alcohol dependence syndrome is the compulsion for, tolerance to, and/or inability to control alcohol consumption.

More preferably, the disorder or symptom associated with alcohol dependence syndrome is anxiety.

More preferably, the disorder or symptom associated with alcohol dependence syndrome is depression.

One of the main conclusions of the examples of the present invention has been that pre-treatment with OEA affected the expression and signaling of the innate immune receptors TLR4 under alcohol intoxication conditions. Therefore, in another preferred embodiment of this aspect of the invention, the use is preventive and the administration of the compound of the invention is performed before the intake of alcohol.

In another preferred embodiment of this aspect, the use is preventive and the administration of the compound of the invention is performed during the intake of alcohol.

The examples of the invention also show that ethanol intoxication induced overexpression and activity of caspase-3 in the frontal cortex, which was inhibited by pre-treatment with OEA. This discovery reveals a mechanism that has not been described until now that OEA can be used to protect the brain.

The data provided by the behavioral experiments is of particular interest, and it indicates that OEA can regulate negative aspects at the behavioral level associated with early stages of alcohol withdrawal. OEA thereby showed antidepressant-like properties in the forced swim test 48 h after alcohol abuse and a tendency to counteract the anxiety induced by alcohol 24 hours after intoxication. The anxiety modulation pattern was less pronounced 12 days after the treatments.

Therefore, another preferred embodiment relates to the use of the compounds of the invention in the production of a medicinal product for the treatment of alcohol-induced anxiety in early stages of alcohol withdrawal.

The compounds of the invention regulate multiple physiological adaptations after ethanol abuse, including the reduction of ethanol self-consumption and relapse, the ethanol-induced neuroinflammation and brain damage, or the attenuation of withdrawal symptoms after the intake of ethanol. Preferably the compounds of the invention are administered before or during alcohol abuse.

In the present invention, “alcohol use or consumption disorders” describe a wide variety of conditions ranging from symptoms caused by alcohol intoxication or pathological inebriation, to those which are associated with alcohol dependence syndrome. They are all classified in ICD-10, section F. 10. Mental and behavioral disorders due to the use of alcohol, and include:
F10.0 Acute intoxication
F10.1 Harmful use
F10.2 Dependence syndrome
F10.3 Withdrawal state
F10.4 Withdrawal state with delirium
F10.5 Psychotic disorder
F10.6 Amnesic syndrome
F10.7 Residual and late-onset psychotic disorder
F10.8 Other mental and behavioral disorders
F10.9 Unspecified mental and behavioral disorder

Veisalgia, commonly known as hangover, is a set of malaise symptoms which may be suffered after an excessive consumption of alcoholic beverages, although it is not enough to become a deep coma and subsequent death due to respiratory depression. It manifests as a set of the following symptoms:
Slight amnesia or memory loss of what occurred during the binging episode.
Gastric disturbances: vomiting almost always, and more rarely diarrhea caused by alcohol causes erosion of the gastric mucosa and loss of intestinal villi.
Cephalea or headache, which is caused by dehydration of the meninges, dilation of the blood vessels and drop in glucose (blood sugar).
Orthostatism and intense thirst, generated as a response of the body to dehydration caused by alcohol degradation.
Abdominal and muscle pain, which translates into a feeling of weakness.
Possible flatulence.
Dulling of the senses.

In this specification, “alcohol” is mainly understood to mean, without limitation, alcoholic beverages containing ethanol. Other alcohols that cause the same symptoms after intake are also possible.

The compound or compounds of the invention, as previously described, can therefore be used in the production of a medicinal product for the prevention, relief, and/or treatment of alcoholism or of a disease or pathological condition caused by the intake of alcohol in a mammal.

In a preferred embodiment of this aspect of the invention, the disease caused by the excessive intake of alcohol is alcohol acute intoxication.

In another preferred embodiment of this aspect of the invention, the disease caused by the excessive intake of alcohol are mental and behavioral disorders (F.10 according to the ICD-10 classification). In another more preferred embodiment, the mental and behavioral disorder is a psychiatric syndrome associated with alcoholism. Even more preferably, the mental and behavioral disorder is anxiety. In another even more preferred embodiment of this aspect of the invention, the mental and behavioral disorder is depression.

### Pharmaceutical Composition of the Invention

A second aspect of the invention relates to the use of a pharmaceutical composition comprising at least one compound of the invention, or a tautomer, a pharmaceutically acceptable salt, a derivative or a prodrug thereof, or any of their combinations, in the production of a medicinal product for the prevention, relief, and/or treatment of alcohol use or consumption disorders in a mammal.

Alternatively, it relates to pharmaceutical composition comprising at least one compound of the invention or any of its salts, preferably any pharmaceutically acceptable salt, esters, tautomers, polymorphs, pharmaceutically acceptable hydrates, or an isomer, prodrugs, derivatives, solvates, or analogues, a derivative or a prodrug thereof, or any of their combinations, for use for the prevention, relief, and/or treatment of alcohol use or consumption disorders in a mammal. In a preferred embodiment of this aspect, the composition of the invention further comprises a pharmaceutically acceptable carrier, an excipient and/or a pharmaceutically acceptable vehicle.

In another preferred embodiment of the first aspect of the invention, the alcohol use or consumption disorders are selected from alcohol intoxication or pathological inebriation, and alcohol dependence syndrome.

In a more preferred embodiment, the alcohol use or consumption disorder is alcohol intoxication or pathological inebriation.

In another even more preferred embodiment, the alcohol use or consumption disorders (or symptoms) are selected from the list consisting of neuroinflammation, neurotoxicity, neuronal death, liver damage, veisalgia, or any of their combinations.

In a more preferred embodiment, the alcohol use or consumption disorder is alcohol dependence syndrome. In an even more preferred embodiment, the alcohol use disorders (or symptoms) are selected from the list consisting of neuroinflammation, neurotoxicity, neuronal death, liver damage, veisalgia, anhedonia, compulsion for, tolerance to, and inability to control alcohol consumption, withdrawal comprising anxiety and depression, or any of their combinations.

More preferably, the alcohol consumption disorder (or symptom) associated with alcohol dependence syndrome is neuroinflammation, neurotoxicity, neuronal death, veisalgia, and/or liver damage, or any of their combinations.

More preferably, the alcohol consumption disorder (or symptom) associated with alcohol dependence syndrome is anhedonia.

More preferably, the alcohol consumption disorder (or symptom) associated with alcohol dependence syndrome is the compulsion for, tolerance to, and/or inability to control alcohol consumption.

More preferably, the alcohol consumption disorder (or symptom) associated with alcohol dependence syndrome is anxiety.

More preferably, the alcohol consumption disorder (or symptom) associated with alcohol dependence syndrome is depression.

In another preferred embodiment of the first aspect of the invention, the use is preventive and the administration of the compound of the invention is performed before the intake of alcohol.

In another preferred embodiment of this aspect, the use is preventive and the administration of the compound of the invention is performed during the intake of alcohol.

In another preferred embodiment of this aspect, the use is preventive and the administration of the compound of the invention is performed before or during the intake of alcohol.

The composition of the invention may comprise a compound of the invention as the only active ingredient. In another preferred embodiment, the pharmaceutical composition further comprises another active ingredient.

The pharmaceutically acceptable adjuvants and vehicles which can be used in said compositions are the adjuvants and vehicles known by those skilled in the art and commonly used in the production of therapeutic compositions.

In the sense used in this description, the expression “therapeutically effective amount” refers to the amount of the agent or compound capable of developing the therapeutic action determined by its pharmacological properties, calculated to produce the desired effect, and it will generally be determined, among others, by the characteristics of the compounds, including the age and condition of the patient, the seriousness of the disturbance or disorder, and the administration route and frequency.

The compounds described in the present invention, their salts, prodrugs, and/or solvates, as well as the pharmaceutical compositions which contain them can be used together with other additional drugs or active ingredients to provide a combination therapy. Said additional drugs can be part of the same pharmaceutical composition, or they can alternatively be provided in the form of a separate composition for the administration thereof at the same time or a different time with respect to the pharmaceutical composition comprising a compound of formula (I), or a salt, prodrug, or solvate thereof.

Therefore, in another preferred embodiment the pharmaceutical composition further comprises another active ingredient. More preferably, the active ingredient is selected from the list consisting of: vitamin E, vitamin C, betaine, N-acetylcysteine, ursodeoxycholic acid, resveratrol, hydroxytyrosol, lycopene, and other antioxidants, nanoelectrolytes, minerals, probiotics, paracetamol, ibuprofen, vitamin B12, caffeine, or any of their combinations.

As it is used herein, the term “active ingredient”, “active substance”, “pharmaceutically active substance”, or “pharmaceutically active ingredient” means any component which potentially provides a pharmacological activity or another different effect in the diagnosis, cure, mitigation, treatment, or prevention of a disease, or which affects the structure or function of the body of humans of other animals. The term includes those components which promote a chemical change in the production of the drug and are present therein in a modified form envisaged for providing the specific activity or effect.

Another aspect of the invention relates to a dosage form, hereinafter dosage form of the invention, comprising the compound of the invention or the composition of the invention.

In this specification, “dosage form” is understood to mean the mixture of one or more active ingredients with or without additives having physical characteristics for the suitable dosage, conservation, administration, and bioavailability thereof.

In another preferred embodiment of the present invention, the compositions and dosage forms of the invention are suitable for oral administration, in solid or liquid form. The possible forms for oral administration are tablets, capsules, syrups, or solutions and they may contain conventional excipients known in the pharmaceutical field, such as binding agents (e.g. syrup, acacia, gelatin, sorbitol, tragacanth or polyvinylpyrrolidone), fillers (e.g. lactose, sugar, corn starch, calcium phosphate, sorbitol, or glycine), disintegrants (e.g. starch, polyvinylpyrrolidone, or microcrystalline cellulose) or a pharmaceutically acceptable surfactant such as sodium lauryl sulfate. Other dosage forms can be colloidal systems, among which nanoemulsions, nanocapsules, and polymeric nanoparticles are included.

The compositions for oral administration can be prepared by conventional Pharmaceutical technology methods, such as mixture and dispersion. The tablets can be coated following methods known in the pharmaceutical industry.

The compositions and dosage forms can be adapted for parenteral administration as sterile solutions, suspensions, or lyophilisates of the products of the invention, using the suitable dose. Suitable excipients, such as pH buffering agents or surfactants, can be used.

The previously mentioned formulations can be prepared using conventional methods, such as those described in the Pharmacopoeias of different countries and in other reference texts.

As it is used in this memory, the term “medicinal product” refers to any substance used for the prevention, diagnosis, relief, treatment, or cure of diseases in humans and animals.

The administration of the compounds, compositions, or dosage forms of the present invention can be performed by means of any suitable method, such as the intravenous infusion and oral, topical, or parenteral administration routes. Oral administration is preferred due to patient convenience and the chronic nature of the diseases to be treated.

The administered amount of a compound of the present invention will depend on the relative efficacy of the chosen compound, the seriousness of the disease to be treated, and the weight of the patient. However, the compounds of this invention will be administered one or more times a day, for example 1, 2, 3, or 4 times daily, with a total dose between 0.1 and 1000 mg/Kg/day. It is important to take into account that it may be necessary to introduce variations in the dose, depending on patient age and condition, as well as modifications in the administration route.

The compounds and compositions of the present invention can be used together with other medicinal products in combined therapies. The other drugs can be part of the same composition or part of another different composition, for administration at the same time or at different times.

The compound or compounds and compositions of the invention, as previously described, can therefore be used in the production of a medicinal product for the prevention, relief, and/or treatment of alcoholism or of a disease or pathological condition caused by the intake of alcohol in a mammal.

### Food Composition

A third aspect of the invention relates to a food composition, a nutraceutical composition, or a “medical food” type composition, hereinafter food composition of the invention, comprising at least one of the compounds of formula (I), formula (II) or formula (III).

The preferred food compositions are selected from the list consisting of: isotonic beverages, preparations based on electrolytes, juices, milk, yogurt, cheese, fermented milk, flavored milk drink, soy milk, pre-cooked grains, bread, cakes, butter, margarine, sauces, oils for frying, vegetable oils, corn oil, olive oil, soybean oil, palm oil, sunflower oil, cottonseed oil, condiments, dressings for salads, fruit juices, syrups, desserts, glazes and fillings, soft frozen products, candies, gums, thistle compositions, and intermediate foods. The food composition of the invention can be a nutritional or dietary supplement. In another preferred embodiment, the nutritional or dietary supplement comprises a sterile composition which contains the compound of the invention, preferably provided with a coating resistant to gastric acids, being a delayed-release composition. In another preferred embodiment, the food composition, including the compound of the invention and/or the nutritional or dietary supplement, comprises suitable “carriers” such as diluents, adjuvants, excipients or vehicles with which the compound of the invention is administered. Appropriate suitable excipients include, without limitation, starch, glucose, fructose, lactose, sucrose, gelatin, malt, rice, flour, calcium sulfate, silica gel, sodium stearate, glycerol monostearate, talc, sodium chloride, skim milk powder, glycerol, propylene, glycol, water, ethanol, and the like. Such nutritional supplements can be used to control liver problems and help to maintain the health or a healthy lifestyle of a mammal, preferably a human being.

A fourth aspect of the invention relates to the use of the food composition of the invention for the prevention, relief, and/or treatment of alcohol use or consumption disorders in a mammal, preferably a human.

In another preferred embodiment of the first aspect of the invention, the alcohol use or consumption disorders are selected from alcohol intoxication or pathological inebriation, and alcohol dependence syndrome.

In another more preferred embodiment, the alcohol use or consumption disorder is alcohol intoxication or pathological inebriation.

In another even more preferred embodiment, the alcohol use or consumption disorders (or symptoms) are selected from the list consisting of neuroinflammation, neurotoxicity, neuronal death, liver damage, veisalgia, or any of their combinations.

In another more preferred embodiment, the alcohol consumption disorder is alcohol dependence syndrome. In an even more preferred embodiment, the alcohol use disorders (or symptoms) are selected from the list consisting of neuroinflammation, neurotoxicity, neuronal death, liver damage, veisalgia, anhedonia, compulsion for, tolerance to, and inability to control alcohol consumption, withdrawal comprising anxiety and depression, or any of their combinations.

More preferably, the alcohol consumption disorder (or symptom) associated with alcohol dependence syndrome is neuroinflammation, neurotoxicity, neuronal death, veisalgia, and/or liver damage, or any of their combinations.

More preferably, the alcohol consumption disorder (or symptom) associated with alcohol dependence syndrome is anhedonia.

More preferably, the alcohol consumption disorder (or symptom) associated with alcohol dependence syndrome is the compulsion for, tolerance to, and/or inability to control alcohol consumption.

More preferably, the alcohol consumption disorder (or symptom) associated with alcohol dependence syndrome is anxiety.

More preferably, the alcohol consumption disorder (or symptom) associated with alcohol dependence syndrome is depression.

In another preferred embodiment of the first aspect of the invention, the use is preventive and the administration of the compound of the invention is performed before the intake of alcohol.

In another preferred embodiment of this aspect, the use is preventive and the administration of the compound of the invention is performed during the intake of alcohol.

The food composition of the invention is used for the improvement of physiological functions, for the prevention, treatment, or improvement of the quality of life in alcohol use disorders.

As it is understood to mean in the present invention, the term “treatment” refers to controlling the effects caused as a consequence of a disease or pathological condition of interest in a subject (preferably a mammal, and more preferably a human) including:
(i) inhibiting the disease or pathological condition, i.e., stopping its development;
(ii) relieving the disease or pathological condition, i.e., causing the abatement of the disease or pathological condition or the symptomatology thereof;
(iii) stabilizing the disease or pathological condition.

As it is understood to mean in the present invention, the term “prevention” consists of avoiding the occurrence of the disease, i.e., avoiding the disease or pathological condition from occurring in a subject (preferably a mammal, and more preferably a human), particularly when said subject has a predisposition for the pathological condition.

Therefore, the nutraceutical composition of the invention is useful for the prevention, relief, or treatment of alcoholism or of a disease or pathological condition caused by the intake of alcohol in a mammal. Alternatively, it relates to the use of the food composition of the invention for the prevention and/or treatment of alcoholism or of a disease or pathological condition caused by the intake of alcohol in a mammal.

Throughout the description and claims, the word “comprises” and variants thereof do not seek to exclude other technical features, additives, components, or steps. For those skilled in the art, other objects, advantages, and features of the invention will be inferred in part from the description and in part from putting the invention into practice. The following examples and drawings are provided by way of illustration and are not meant to limit the present invention.

### CLAUSES

1. Method for the prophylactic or preventive treatment of alcohol consumption disorders comprising the use of a nutraceutical or medicinal composition comprising a compound of general formula (I)

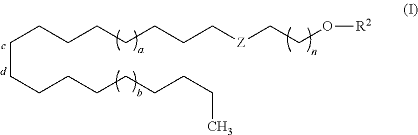

or any of its pharmaceutically acceptable salts, esters, tautomers, solvates, and hydrates, or any of their combinations;
wherein
n is an integer between 0 and 5;
a and b are determined by the following formula: 0≤(a+b)≤4; and
Z is a group which is selected from —C(O)N(R⁰)—; —(R⁰)NC(O)—; —(O)CO—; O; NR⁰; and S, and where R⁰ and R² are independently selected from the group consisting of a substituted or unsubstituted alkyl, hydrogen, a substituted or unsubstituted (C1-C6) alkyl, a substituted or unsubstituted (C1-C6) acyl, homoalkyl, and aryl, and wherein up to eight hydrogen atoms of the compound can be substituted by methyl or a double bond, and the molecular bridge existing between c and d can be saturated or unsaturated.
   2. The method according to the preceding clause, wherein the compound presents the formula (II),

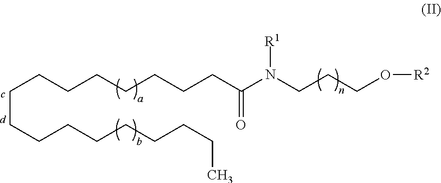

or any of its pharmaceutically acceptable salts, esters, tautomers, solvates, and hydrates, or any of their combinations;
wherein
n is an integer between 0 and 4;
a and b are determined by the following formula: 0≤(a+b)≤3; and
R¹ and R² are independent members which are selected from the group consisting of a substituted or unsubstituted (C1-C6) alkyl, a substituted or unsubstituted (C1-C6) acyl, and wherein up to eight hydrogen atoms of the compound can be substituted by methyl or a double bond, and the molecular bridge existing between c and d can be saturated or unsaturated.
   3. The method according to any of clauses 1-2, wherein a=1 and b=1.
   4. The method according to any of clauses 1-3, wherein n=0 or 1.
   5. The method according to any of clauses 2-4, wherein R¹ and R² are hydrogens (H).
   6. The method according to any of clauses 1-5, wherein the bridge between carbon c and carbon d is a double bond.
   7. The method according to any of clauses 1-6, wherein the compound of formula (I) is an acylethanolamide.
   8. The method according to any of clauses 1-7, wherein the acylethanolamide is selected from the list consisting of oleoylethanolamide (OEA), palmitoylethanolamide (PEA), stearoylethanolamide (SEA), or any of their combinations.
   9. The method according to clause 8, wherein the acylethanolamide is the oleoylethanolamide (OEA) of formula (III)

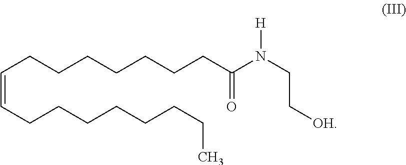 [SMILES: CCCCCCCC/C=C\CCCCCCCC(=O)NCCO]

10. The method according to any of clauses 1-9, wherein said alcohol consumption disorders are selected from the list consisting of: alcohol intoxication or pathological inebriation, and alcohol dependence syndrome.
11. The method according to clause 10, wherein the alcohol consumption disorder is alcohol intoxication or pathological inebriation.
12. The method according to any of clauses 1-10, wherein the alcohol consumption disorder is alcohol dependence syndrome.
13. The method according to any of clauses 1-12, wherein said method is prophylactic or preventive and the administration of the compound is performed prior to the intake of alcohol.
14. The method according to any of clauses 1-12, wherein said method is prophylactic or preventive and the administration of the compound of the invention is performed during the intake of alcohol.
15. The method of according to any of clauses 1 to 14, further comprising a second active ingredient selected from the list consisting of ibuprofen, paracetamol, vitamin B12, and caffeine, or any of their combinations.
16. The method according to any of clauses 1 to 15, wherein the composition is a nutraceutical composition and is selected from the list comprising or consisting of preparations based on electrolytes and/or isotonic beverages, natural juices, thistle teas, caffeine preparations, or any of their combinations.

### EXAMPLES OF THE INVENTION

### Example 1. Materials and Methods

Animals

87 male Wistar rats (from Harlan, Spain) weighing 250-300 g were used. The animals were housed in groups (n=4-6) in a 12-hour reverse light/dark cycle in normal temperature and humidity conditions. Standard food (A04 SAFE, Scientific Animal Food and Engineering, Augy, France) and ad libitum tap water were available in the cage. All the animals were kept under constant conditions for 10 days before the experiments.

All the experimental protocols complied with the guidelines of the Animal Protection Committee of the Universidad Complutense of Madrid, following European legislation (2010/63/EU).

Treatment of Ethanol Intoxication

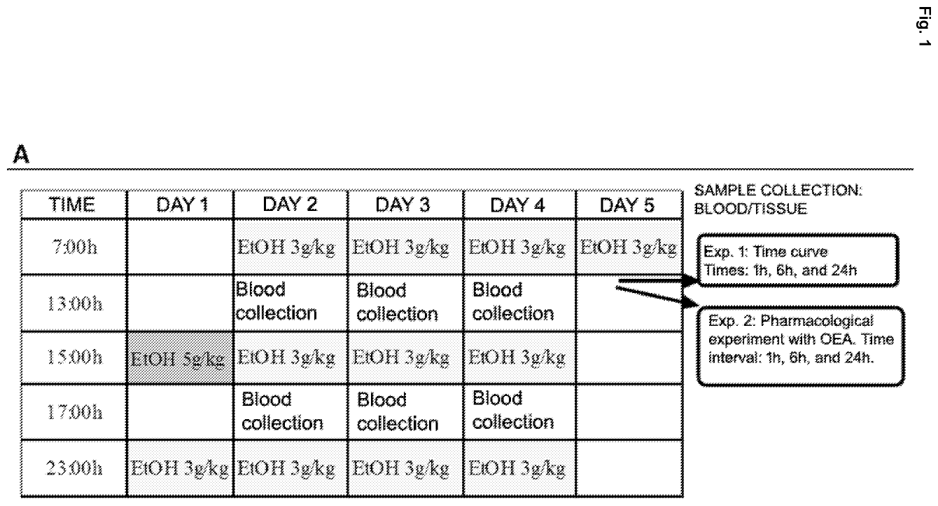

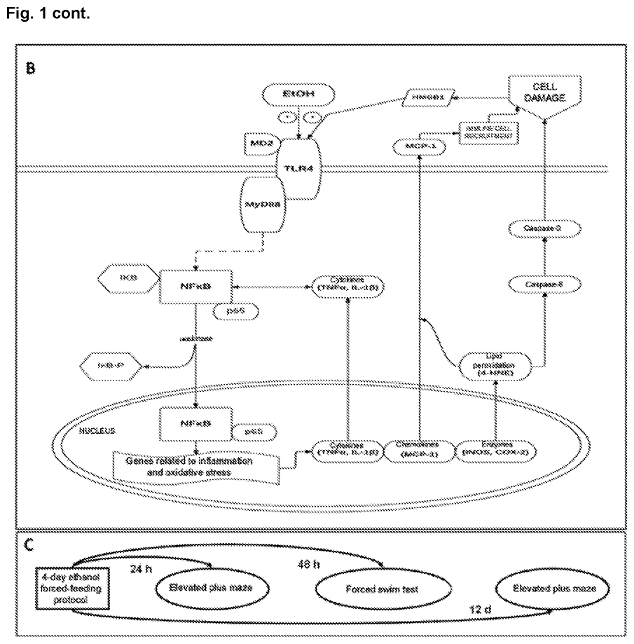

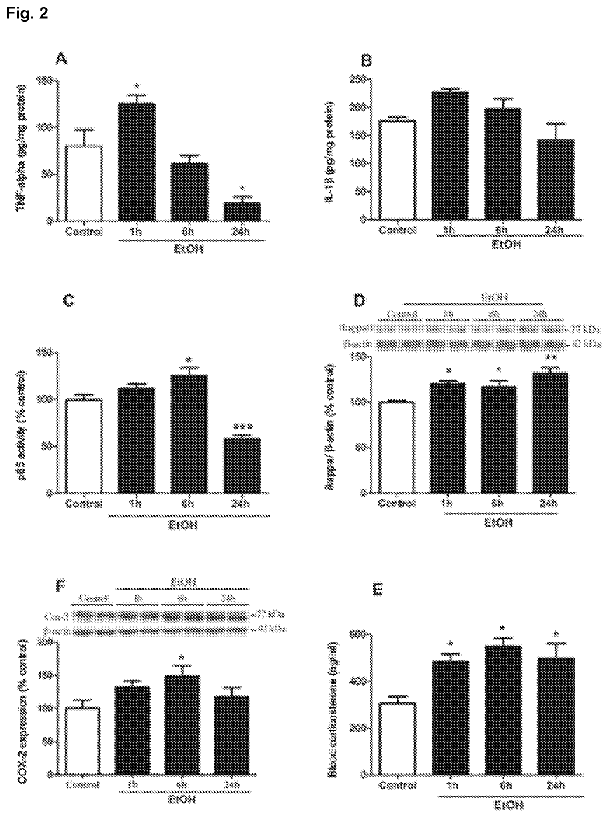

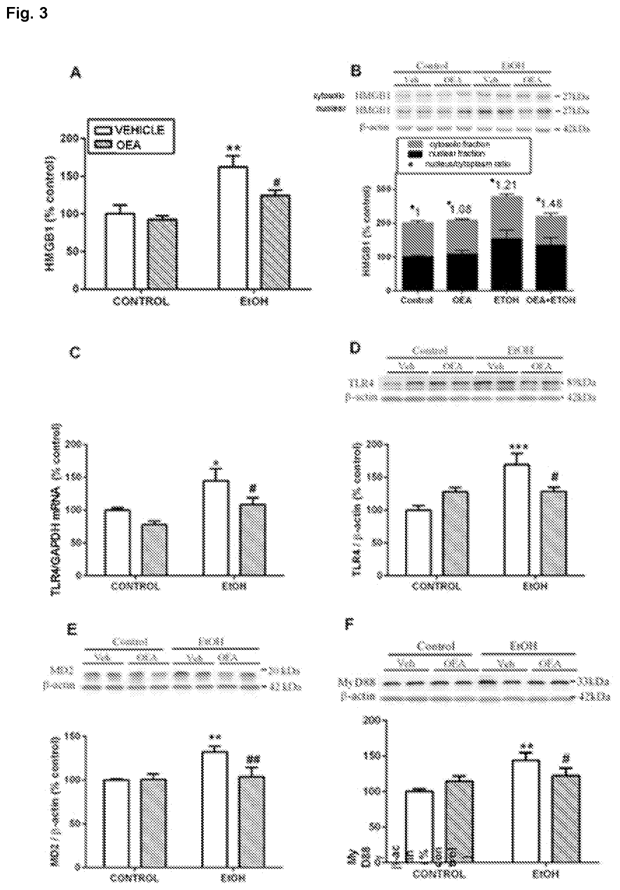

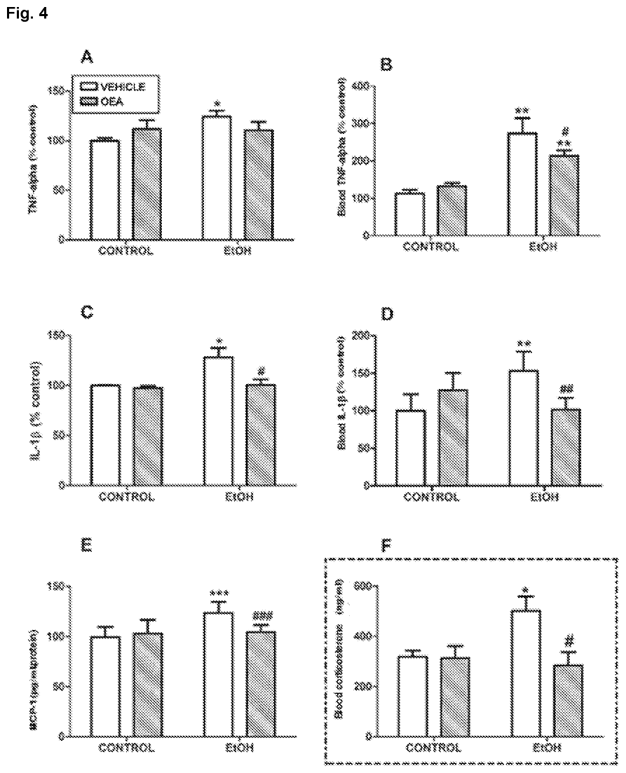

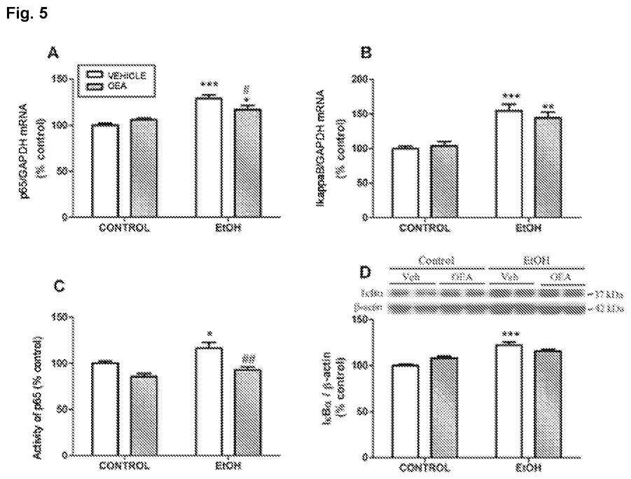

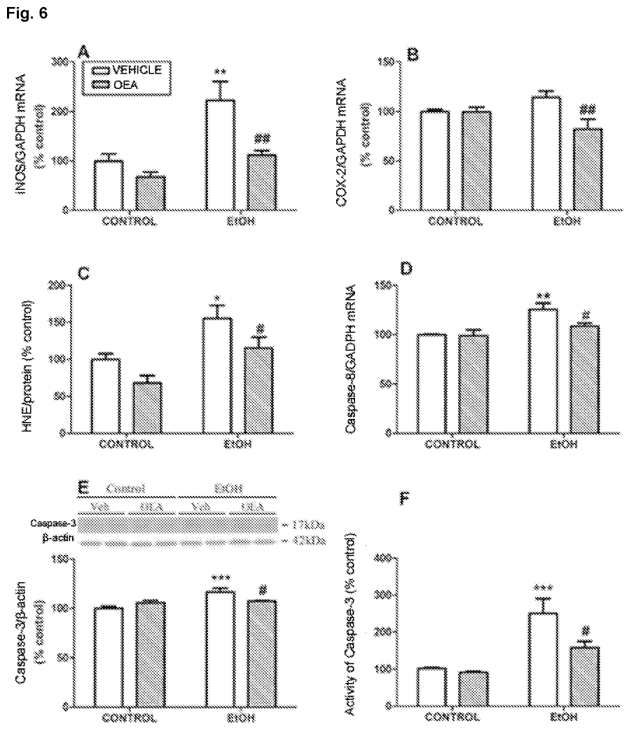

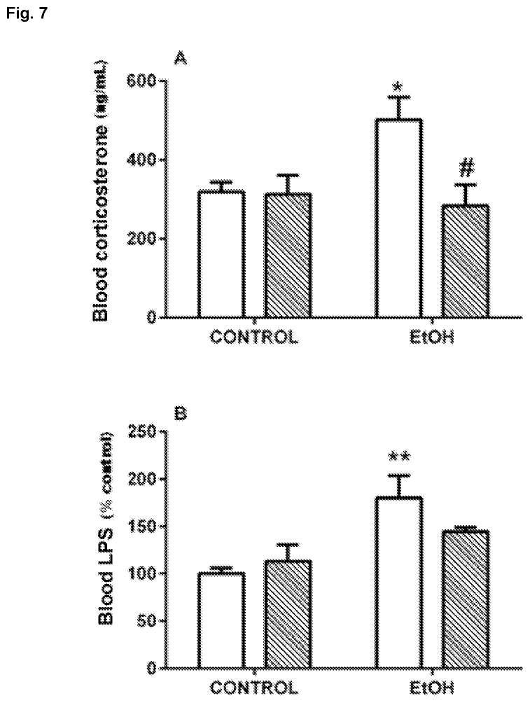

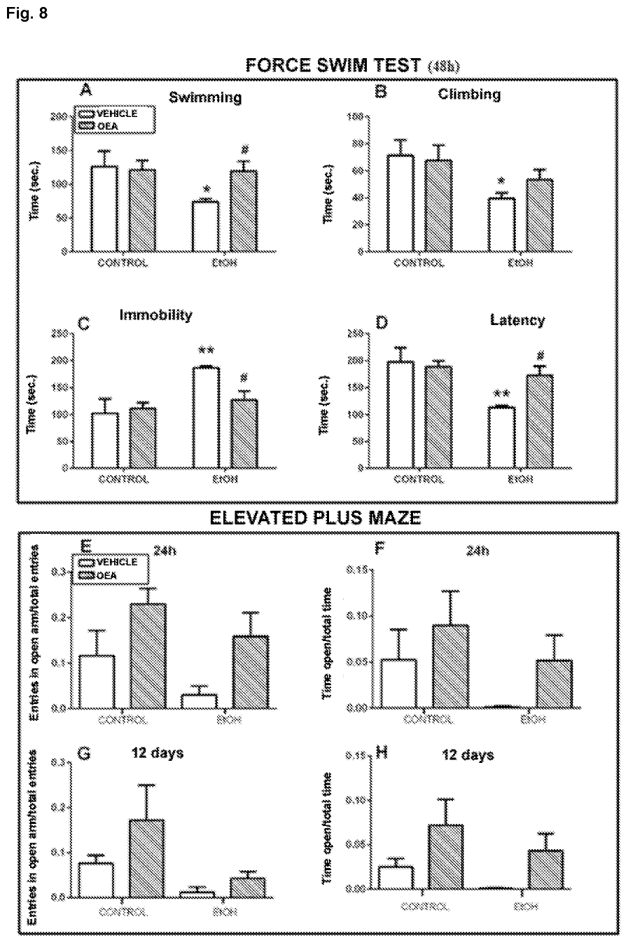

The animals were treated with intragastric (i.g.) ethanol 3 times a day using an i.g. cannula (16G needle, Fisher Scientific, Waltham, Mass., USA), following a protocol based on a slightly modified standard 4-day alcohol intoxication pattern (Bernier et al., 2002a, b) (FIG. 1A). The ethanol doses were administered every 8 h for a total time of 4 days. The rats treated with ethanol received an initial loading dose of 5 g/kg of ethanol in a 30% solution (w/v) and then a maximum maintenance dose of 3 g/kg, which were determined based on blood ethanol levels (BEL). This repeated standard alcohol intoxication paradigm kept BEL intoxication relatively constant in a range of sedation/ataxia (190 to 430 mg/dl, Table 3), according to the 6-point alcohol intoxication behavior scale (Majchrowicz, 1975). The mean dose of ethanol/rat/day (days 1-4) was 7.06 g/kg, similar to other studies (Obernier et al., 2002a, b).

TABLE 3
Blood ethanol levels.

| | Blood | | | | |
|---|---|---|---|---|---|
| | collection | Day 2 | Day 3 | Day 4 | |
| TREATMENT | time | BEL (g/dL) | BEL (g/dL) | BEL (g/dL) | |
| Vehicle + EtOH | 13:00 | 193.29 ± 38.22 | 341.73 ± 32.48** | 303.32 ± 37.17* | N = 10 |
| | 17:00 | 274.96 ± 27.17 | 422.35 ± 18.00*** | 430.31 ± 19.72*** | N = 10 |
| OEA + EtOH | 13:00 | 217.14 ± 41.58 | 342.57 ± 40.25* | 320.68 ± 36.63 | N = 9 |
| | 17:00 | 321.88 ± 34.08 | 404.17 ± 27.65 | 429.76 ± 29.25* | N = 9 |

The data represents BEL 2 h before and 2 h after the 15:00 h administration of alcohol to animals in a vehicle or pretreated with OEA. There were differences in BEL between treatment day 2 and treatment days 3 or 4 in both groups. Pre-treatment with OEA did not modify the BEL reached during treatment days 2-4 with alcohol intoxication neither before nor after ethanol force-feeding. The results are expressed as means ± S.E.M. Different from day 2 in the same treatment group:
*p < 0.05;
**P < 0.001.
[EtOH: ethanol; OEA: oleoylethanolamide; BEL: blood ethanol levels].

Determination of Blood Ethanol Levels

To confirm intoxication, BEL was determined in blood samples taken from the tail 120 minutes before and after the second i.g. administration of ethanol of the day, which was done at 3 pm by means of the use of electrochemical detection of an enzymatic reaction with an AM1 alcohol analyzer (Analox Instruments, London, United Kingdom).

Experimental Design, Administration of the Drug and Collection of Tissue/Plasma

### 1. Time Curve of the Main Neuroinflammatory Changes Induced by the Alcohol Intoxication Protocol (FIG. 1A)

The animals (n=22) subjected to 4 days of alcohol intoxication treatment by means of feeding with a gavage (see FIG. 1A) were sacrificed 1 h, 6 h, and 24 h after the last administration of ethanol using a lethal dose of pentobarbital sodium (300 mg/kg, ip, Dolethal®, Spain). The brains were separated from the cranium, and the meninges and blood vessels were carefully disposed off. The frontal cortex was extirpated and frozen at −80° C. until the assay. Blood was collected by cardiac puncture using trisodium citrate (3.15% w/v) as an anticoagulant. The plasma was obtained by blood centrifugation (2000 g) for 15 min at 4° C. and was stored at −20° C. until the determinations were made.

This first experiment allowed observing the moment of expression of the main inflammation markers. Accordingly, a second experiment was designed in which brain tissue samples were collected in a range between 2-4 h after the last ethanol intoxication. This interval of between 1 and 6 hours was chosen for detecting both the first changes in the inflammatory parameters, for example the overexpression of TNF-α, and the activation of subsequent markers, such as the expression of COX-2 (FIG. 2).

### 2. Effects of the Repeated Oral Administration of OEA on Disturbances of Neuroinflammatory Markers Induced by Exposure to Alcohol Intoxication (FIG. 1A).

In this second experiment (n=40 animals), the objective was to test the anti-inflammatory/neuroprotective effects of OEA on the TLR4 signaling cascade (FIG. 1B) in a model of alcohol abuse-induced intoxication.

OEA (5 mg/kg, i.p., except initial dose of 10 mg/kg i.p.; synthesized as described in Giuffrida et al., 2000) was dissolved in the vehicle (5% Tween-80 in saline solution) and injected 10 minutes before each of the administrations of ethanol by an intragastric gavage. The anti-inflammatory activity of OEA at 10 mg/kg per intraperitoneal route in the frontal cortex before the injection of LPS (Sayd et al., 2014) was previously referred to. In this study, OEA is repeatedly injected as a pre-treatment before each administration of ethanol. The doses of OEA chosen in this study are in the range of doses tested as being pharmacologically active (5-20 mg/kg, i.p.) (Bilbao et al., 2015).

A time curve (n=18) was also established with OEA (3 h, 6 h, and 24 h) to rule out any effect in the control animals.

### 3. Effects of the Repeated Administration of OEA on Depressive-Like Behavior and Anxiety During Early Ethanol Withdrawal (FIG. 1C).

In this third experiment (n=25), the treatment with ethanol intoxication and pre-treatment with OEA applied in experiment 2 was repeated, and the animals were subjected to elevated plus maze test 24 h and 12 days after the last forced administration of ethanol and to the forced swim test 48 h after the ethanol intoxication protocol.

Preparation of Nuclear and Cytosol Extracts

The nuclear and cytosol protein extracts were obtained as described in earlier publications (Sayd et al., 2014).

Western Blot Analysis

The frontal cerebral cortexes were homogenized by sonication in 400 μl of PBS (pH=7.4) mixed with a protease inhibitor cocktail (Complete, Roche®, Madrid, Spain), followed by centrifugation at 12,000 g for 10 min at 4° C. Homogenates with adjusted protein levels were mixed with Laemmli sample buffer (BioRad®, CA, USA) which contained β-mercaptoethanol (50 μl/ml of Laemmli) and 1 mg/ml was loaded in electrophoresis gel. The proteins were transferred to a nitrocellulose membrane (Amersham Ibérica®, Spain) with a semi-dry transfer system (Bio-Rad®, CA, USA), were incubated with specific primary and secondary antibodies (see complementary information) and developed by means of the ECL™ kit (Amersham Ibérica®, Spain). The autoradiograph was quantified by densitometry (NIH ImageJ® software, National Biosciences, Lincoln, Nebr. USA) and expressed as optical density (OD). In all the Western blot analyses, protein β-actin was used as a loading control.

Real-time Polymerase Chain Reaction (RT-PCR) Analysis

Total cytoplasmic RNA was prepared from frontal cortex samples using TRIZOL® reagent (Invitrogen, Grand Island, N.Y., USA). The aliquots were converted into cDNA using random hexamer primers. The quantitative changes in mRNA levels were estimated by means of RT-PCR, carried out in the presence of the SYBR green primer using a 20-L reaction in a Rotor-Gene (Corbett Research®, Mortlake, NSW, Australia). See the complementary information for the description and additional details of the sequence of the primers used.

HMGB1, Proinflammatory Cytokine (TNF-α, IL-1β), and MCP-1 Chemokine Levels

The levels of soluble HMGB1, TNF-α, IL-1β and MCP-1 were determined by means of a commercially available enzyme-linked immunosorbent assay kit (ELISA) (Elabscience Biotechnology Co., Ltd., China, for HMGB-1; RayBiotech®, GA, USA, for TNF-α, IL-1β, and MCP-1). The nuclear/cytosol HMGB1 fraction was determined by means of Western blot analysis.

Assay of the Transcription Factor NF-κB

The nuclear extracts were used for the determination of the activity of transcription factor NF-kB by means of the use of a kit based on ELISA (Cayman Chemicals©, Tallin, Estonia). The nuclear extracts of the frontal cortex were incubated with specific NF-kB p65 subunit response element probes, and the binding of p65 to its probe was detected using an antibody specific for this subunit. A secondary antibody labeled by means of horseradish peroxidase was added, and the binding was detected by means of spectrophotometry at 450 nm. The measurement was taken according to the manufacturer's instructions. This assay is specific for the activation of p65, and it does not present cross-reactivity with other NF-kB subunits, such as p50. The data was normalized by the amount of total protein.

Lipid Peroxidation

Lipid peroxides are unstable indicators of oxidative stress in cells which break down to form more complex and reactive compounds, such as 4-hydroxynonenal (4-HNE), a natural byproduct of lipid peroxidation. The levels of HNE protein adducts in cerebral cortex lysates were measured using an OxiSelect™ HNE adduct competitive ELISA 96-well kit (Cell Biolabs®, San Diego, Calif., USA).

Measurement of Caspase-3 Activity

Caspase-3 activity was determined using a fluorometric assay kit (Abnova®, Taiwan) according to the manufacturer's protocol. This commercial kit allows a measurement of DEVD-dependent caspase activity.

Corticosterone and LPS Plasma Levels

Corticosterone and LPS were measured in plasma by means of the use of radioimmunoassay (R.I.A.) kits available on the market (Coat-A-Count®, Siemens, La., USA) or colorimetric enzymatic reaction (HyCult Biotech, Uden, Netherlands), respectively.

Protein Assay

Protein levels were measured using the Bradford method based on the principle of protein dye binding (Bradford, 1976).

Behavioral Assays

The rats were subjected to the forced swim test two days after the last ethanol force-feeding (see FIG. 1C for the experimental design), and specific behaviors were recorded with a digital video camera for 5 minutes. These same rats were also subjected to the elevated plus maze test equipped with infrared light photography (Panlab, Barcelona, Spain) 1 and 12 days after the last ethanol force-feeding, where the animals were allowed to freely explore the maze for 5 minutes. The tests were performed 3 h after the start of the dark phase.

Statistical Analysis

The data in the text and figures is expressed as the media ±S.E.M. The data was analyzed by means of a two-way analysis of variance (ANOVA), comparing the factors [alcohol/water] to [OEA/vehicle], followed by a Bonferroni post-hoc test, and by a one-way analysis of variance ANOVA followed by the Newman-Keuls post-hoc test, when required. The BEL data was analyzed by a two-way repeated measure analysis of variance ANOVA followed by a Bonferroni post-hoc test (treatment×daily determination) for the pre- and post-tube force-feeding BEL determinations. The data of the behavioral tests was studied to determine normal conditions with the Kolmogorov-Smirnov test, and then analyzed by parametric two/one-way ANOVA followed by Bonferroni/Newman-Keuls post-hoc tests (forced swim test), or by non-parametric Kruskal-Wallis ANOVA followed by Dunn's post-hoc test for multiple comparisons (elevated plus maze), where appropriate. A value of p≤0.05 was statistically significant. The data was analyzed using GraphPad Prism version 5.04 (GraphPad Software Inc, San Diego, Calif., USA)

### Example 2. Time Curve of Proinflammatory Markers in the Frontal Cortex Induced by Alcohol Intoxication and Increase in Plasma Corticosterone Levels

TNF-α of the frontal cortex was overexpressed 1 hour after the administration of ethanol [55% increase with respect to the control (80.29±17 pg/mg of protein)] and the cytokine content dropped 24 hours after treatment (FIG. 2A; F_{(3, 11)}=9.45; p=0.0002). IL-1β showed a tendency to increase 1 h after exposure to ethanol [29% increase with respect to the control (75.5±6.6 pg/mg of protein)], but the data did not amount to being statistically significant (FIG. 2B).

The expression of NF-kB p65 subunit increased in the nuclear extracts of the frontal cortex 6 h after treatment with ethanol, and dropped 24 h after ethanol intoxication, when compared with the control group (FIG. 2C; F_{(3, 11)}=20.48; p<0.0001). The levels of the NF-kB inhibitory protein, IkappaB-alpha (IκBα), remained high 1 h, 6 h, and 24 h after treatment with alcohol in the cytosol extract of the frontal cortex (FIG. 2D; F_{(3, 13)}=6.73; p=0.0056) and COX-2 enzyme showed overexpression 6 h after the administration of ethanol (FIG. 2E; F_{(3, 13)}=3.69; p=0.04). Finally, exposure to alcohol intoxication raised plasma corticosterone levels 1 h, 6 h, and 24 h after the last administration (FIG. 2F; F_{(3, 15)}=3.91; p=0.03).

Based on this expression pattern of the main proinflammatory markers between 1 and 6 hours after the last administration of ethanol by an intragastric gavage, it was decided to remove brain/plasma samples in a time interval of 2-4 h after the intoxication protocol to study the pharmacological effects of OEA.

### Example 3. Blood Ethanol Levels

BEL reached during the experiment was in the range described by others (Knapp and Crews et al., 1999; Obernier et al, 2002a, b; Crews et al, 2006). It was studied if OEA may modify BEL during days 2-4 of treatment with excessive alcohol (Table 1). The two-way repeated measure ANOVA detected differences in the BEL over the days of the intoxication protocol (F_{(2, 17)}=10.17; p=0.0003; F_{(2, 17)}=15.41; p<0.0001) and demonstrated that pre-treatment with OEA did not change the BEL reached during days 2-4 of treatment with ethanol intoxication, or during the prior force-feeding (F_{(1, 17)}=0.13; p=0.72, ns) or during post-feeding (F_{(1, 17)}=0.18; p=0.67, n.s.). BEL also was determined right before removing the tissue (day 5), being 431.98±34.90 g/dl for the vehicle+ethanol group and 445.82±32.70 g/dl for the OEA+ethanol group (Student t-test, p=0.78, n.s.).

### Example 4. Disturbances of the HMGB1/TLR4 Signaling Pathway and the Effect of Pre-Treatment with OEA in Excessive Ethanol Consumption

For the purpose of analyzing if proinflammatory mediators such as cytokines or NF-κB were secreted by the activation of ethanol-induced innate immune receptors, the influence of ethanol intoxication on the expression of TLR4 and its endogenous activator, HMGB1 cytokine danger signaling, was verified. Furthermore, was verified the co-receptor of TLR4, myeloid differentiation 2 (MD2) protein, and expression of myeloid differentiation 88 (MyD88) factor, which is an adaptor protein of the TLR4 intracellular transduction signaling cascade.

The main effects on the expression of the HMGB1 have been observed with exposure to ethanol and pre-treatment with OEA. The ethanol intoxication-induced increase in HMGB1 (F_{(1, 13)}=19.14, p=0.0008) was prevented by means of pre-treatment with OEA (FIG. 3A; F F_{(1, 13)}=4.58, p=0.05). The analysis of the HMGB1 cytoplasm/nuclear ratio (FIG. 3B) showed that the total increase in the expression of HMGB1 (FIG. 3A) in animals exposed to ethanol can be due to an increase in the cytokine in both nucleus and cytoplasm, with the levels in rats treated with ethanol being kept higher in the nucleus than in the cytoplasm (n/c ratio=1.21). Pre-treatment with OEA reduced the ethanol-induced expression of HMGB1 more efficiently in the cytosol fraction (n/c ratio=1.48), which is indicative of showing activity on the release of HMGB1 from the nucleus into the cytoplasm instead of on a level of synthesis.

Treatment by means of ethanol intoxication shows a main effect both on TLR4 mRNA (FIG. 3C; F_{(1, 17)}=10.08, p=0.005) and on protein expression (FIG. 3D; F_{(1,24)}=10.04, p=0.004), a main effect of OEA on TLR4 mRNA (F_{(1, 17)}=6.08, p=0.0246), and an interaction between exposure to ethanol and OEA (F_{(1, 24)}=9.67, p=0.0048) at TLR4 protein levels. Pre-treatment with OEA was able to reduce the ethanol-induced overexpression of TLR-4 mRNA (FIG. 3C. F_{(3, 17)}=5.86, p=0.0061) and the expression of proteins (FIG. 3D; F_{(3,24)}=8.15, p=0.0006).

Exposure to ethanol by intoxication increases the expression of the MD2 protein (FIG. 3E; F_{(1, 27)}=6.83, p=0.0145), but the mRNA expression data did not reach statistical significance (data not shown; F_{(1, 31)}=3.79, p=0.06, n.s.). The main effect of pre-treatment with OEA (F_{(1, 27)}=4.24, p=0.04) and an interaction (exposure to ethanol by pre-treatment) (F_{(1, 17)}=4.57, p=0.0418) were also found at MD2 protein levels. Pre-treatment with OEA inhibited the ethanol-induced increase in MD2 (FIG. 3E; F_{(3,30)}=5.52, p=0.044).

Ethanol intoxication induced a main effect in MyD88 mRNA (F_{(1,15)}=10.34, p=0.0058; data not shown) and protein expression (FIG. 3F; F_{(1, 30)}=9.55, p=0.0043), and an interaction (exposure to ethanol by pre-treatment) was also found at MyD88 protein levels (F_{(1, 30)}=4.640, p=0.0394). Pre-treatment with OEA inhibited the ethanol-induced increase in the expression of the MyD88 protein (FIG. 3F; F_{(3, 32)}=5.46, p=0.042).

### Example 5. Effects of OEA on Frontal Cortex and Plasma Proinflammatory Cytokines TNF-α and IL-1β and Chemokines MCP-1

The treatment with excess ethanol increased the expression of both TNF-α mRNA and IL-1β mRNA in the frontal cortex, although pre-treatment with OEA did not prevent this effect. The levels of TNF-α protein were increased in rat frontal cortexes under treatment with excess ethanol (F_{(1, 16)}=2.56, p=0.1291) [increase by 24% with respect to the control values (95.2±3.1 pg/mg of protein)] and were not prevented by means of OEA (FIG. 4A; F_{(3, 16)}=0.02, p=0.89, ns). In plasma, a conclusive increase in levels of TNF-α in the animals treated with ethanol (F_{(1, 14)}=44.64, p<0.0001) [141% increase with respect to the control values (113.0±5.1 pg/mg of protein)] and an interaction between the ethanol and pre-treatment with OEA (F_{(1, 14)}=4.85, p=0.045) were observed. One-way ANOVA indicated that pre-treatment with OEA partially prevented the increase in plasma TNF-α observed after ethanol intoxication (FIG. 4B; F_{(3, 17)}=15.59, p<0.0001).

The animals treated with ethanol showed an increase in IL-1β protein levels in the frontal cortex (F_{(1, 16)}=4.497, p=0.0499) [27% increase with respect to the control values (79.31±0.29 pg/mg of protein)], which were prevented by pre-treatment with OEA (FIG. 4C; F_{(1, 16)}=4.22, p=0.05). In plasma, an interaction between pre-treatment with OEA and the administration of ethanol (F_{(1,20)}=18.15, p=0.0004) was observed. Treatment with excess ethanol increased IL-1β plasma levels [53% increase with respect to the control values (85.29±7.8 pg/mg of protein)], an effect which was prevented by OEA (FIG. 4D; F_{(3, 20)}=8.20, p=0.0009).

With respect to MCP-1 levels, there was an interaction between the administration of ethanol and pre-treatment with OEA (F_{(1, 33)}=10.06, p=0.0033) and the main effects of the ethanol (F_{(1,33)}=4.69, p=0.0377) and pre-treatment with OEA (F_{(1, 33)}=12.88, p=0.0011). The treatment with excess ethanol increased MCP-1 levels in the frontal cortex and pre-treatment with OEA prevented this effect (FIG. 4E; F_{(3,33)}=9.92, p<0.0001).

### Example 6. Effect of OEA on the Expression of NF-κB and Activity after Exposure to Ethanol Intoxication

The proinflammatory canonical pathway of NF-kB was examined in frontal cortex nuclear and cytosol extracts after the pharmacological treatments. There was a main effect of the treatment for ethanol intoxication on p65 mRNA levels (FIG. 5A; F_{(1, 33)}=44.14, p<0.0001) and an interaction between treatment with ethanol and pre-treatment with OEA (F_{(1, 33)}=9.10, p=0.0049). One-way ANOVA showed that pre-administration of OEA partially prevented the ethanol-induced increase in p65 mRNA levels (FIG. 5A; F_{(3, 36)}=19.87, p<0.0001). In the activity assay, the main effects of exposure to alcohol (F_{(1, 16)}=6.55, p=0.021) and pre-treatment with OEA (F_{(1, 16)}=16.66, p=0.0009) were observed, with the ethanol-induced increase in the nuclear activity of p65 being counteracted by pre-treatment with OEA (FIG. 5C).

The treatment with excess ethanol also increased the expression of mRNA and the IκBα protein (FIGS. 5 B & D; F_{(1, 35)}=50.64, p<0.0001; F_{(1, 33)}=37.69, p<0.0001) and interacted with pre-treatment with OEA (F_{(1, 33)}=8.81, p=0.0055). Pre-treatment with OEA did not counteract the increase in IκBα mRNA levels and the ethanol-induced protein expression (FIGS. 5B and D; F_{(1, 35)}=0.17, p=0.6848 n.s.; F_{(1,33)}=0.17, p=0.067, p=0.7970, n.s., respectively).

### Example 7. Effect of Pre-Treatment with OEA on the Expression of COX-2 and iNOS mRNA and Lipid Peroxidation after Exposure to Ethanol Intoxication

The activation of NF-κB results in the production of inflammatory mediators such as the inducible enzymes iNOS and COX-2. The model of ethanol intoxication used in this study produced a conclusive increase in the expression of iNOS mRNA (F_{(1, 17)}=11.87, p=0.0031) which was entirely prevented by pre-treatment with OEA (FIG. 6A; F_{(1, 17)}=8.71, p=0.0089). The maximum expression of COX-2 observed after treatment with ethanol in the time-course experiment was carried out 6 h after the end of the intoxication protocol (again see FIG. 2F), which may explain the absence a significant effect of ethanol on the regulation of COX-2 mRNA in the second experiment, in which brain samples were collected between 2-4 h after the end of the last administration of ethanol (FIG. 6B; F_{(1, 32)}=0.07, p=0.7975, ns). However, the 2-way ANOVA detected a main effect of pre-treatment with OEA (F_{(1, 32)}=7.17, p=0.0011) and an interaction (ethanol×OEA) (F_{(1, 32)}=6.94, p=0.0129). The one-way ANOVA showed that OEA reduced the levels of COX-2 mRNA with ethanol, but not in the animals treated with the vehicle (FIG. 6B; F_{(3,32)}=4.26, p=0.0123).

As a result of an over-activation of the proinflammatory pathways, the cell mechanisms can be activated, inducing the formation of lipid peroxides which will affect cell viability. These experiments verified the formation of the HNE adduct, a reactive compound formed by the decomposition of lipid peroxides. An increase in the formation of HNE in the frontal cortex of the animals exposed to treatment for ethanol intoxication (FIG. 6C; F_{(1, 15)}=14.56, p=0.0017) and an effect of pre-treatment with OEA (F_{(1, 15)}=7.21, p=0.017) were observed. OEA reduced the formation of HNE, with the significant effect being observed only in the animals treated with ethanol (FIG. 6C; (F_{(3, 18)}=7.03, p=0.0036).

### Example 8. Effect of OEA on Proapoptotic Caspase-3 and Caspase-8 after Treatment for Ethanol Intoxication

The aim was to study if the induction of the TLR4/NF-κB signaling cascade by ethanol resulted in neuronal damage. The expression and activity of caspase-3 proapoptotic enzyme in the frontal cortexes of the animals undergoing the different pharmacological treatments, and the overexpression of caspase-8, which acts like an intermediate caspase in the TLR4-MyD88/caspase-8/caspase-3 signaling pathway (Liu et al., 2013), were verified.

Caspase-8 mRNA was over-expressed after ethanol intoxication (F_{(1, 15)}=13.37, p=0.023), and pre-treatment with OEA reversed this effect (FIG. 6D; F_{(3, 18)}=6.95, p=0.037). Caspase-3 mRNA was over-expressed with alcohol, but OEA was unable to prevent this effect. With respect to the caspase-3 protein levels (FIG. 6E), an interaction between exposure to alcohol and pre-treatment with OEA (F_{(1, 15)}=7.95, p=0.0129) and a main effect of the ethanol (F_{(1, 15)}=12.30, p=0.0032) was observed. The one-way ANOVA indicated that pre-treatment with OEA prevented the ethanol-induced overexpression of caspase-3 (FIG. 6E; F_{(3, 18)}=7.29, p=0.0031). Furthermore, treatment with ethanol intoxication induced an increase in the activity of caspase-3 in the frontal cortex (F_{(1, 32)}=20.98, p<0.0001), which was reverted by pre-treatment with OEA (FIG. 6F; F_{(1, 32)}=4.744, p=0.0369).

### Example 9. Effect of OEA on Plasma Corticosterone and LPS Levels

To verify the effects on the axis (HPA), the influence of OEA on the ethanol-induced increase in corticosterone levels was tested (FIG. 7A). The data showed a significant effect of the treatment (F_{(1, 29)}=5.44, p=0.00268) and an interaction between ethanol intoxication and pre-treatment with OEA (F_{(1, 29)}=4.77, p=0.0373). An increase in corticosterone levels was observed in the animals exposed to treatment for ethanol intoxication, which was prevented entirely by means of pre-treatment with OEA (F_{(3, 29)}=4.21, p=0.0136). OEA did not modify the plasma corticosterone levels in the control animals (FIG. 7A).

Furthermore, the effect of pre-treatment with OEA on intestinal permeability to endotoxins was studied by means of checking plasma LPS levels after treatments. Treatment for ethanol intoxication significantly increases plasma LPS levels by about 80% with respect to the control (F_{(1, 27)}=12.40, p=0.0015). The animals pretreated with OEA and receiving ethanol showed lower plasma LPS levels (44% increase with respect to the control), which value does not differ from the control group or the ethanol group. Two-way ANOVA indicated that there was no significant effect on pre-treatment (F_{(1, 27)}=0.50, p=0.4872) and that there is no interaction between exposure to ethanol and pre-treatment with OEA (F_{(1, 27)}=2.376, p=0.01348).

### Example 10. Effect of OEA on the Control Animals

The OEA time curve in the control animals indicates that OEA did not significantly modify the parameters in the controls. However, given that some trends were observed in comparison with the control levels 3 h after the injection (i.e., TLR4 and p65), it cannot be ruled entirely that OEA may affect control animals in early stages.

### Example 11. Effect of OEA on the Depressive-Like and Anxiety Symptoms

For the purpose of determining the effects of OEA on behaviors related to ethanol during alcohol withdrawal, the forced swim test and the elevated plus maze test were performed to verify depressive-like and anxiety symptoms, respectively. The results showed that ethanol intoxication caused a reduction in swimming and climbing times (FIGS. 8A & B., F_{(1,21)}=3.281, p=0.05; F_{(1.21)}=7.326, p=0.0132, respectively) and an increase in immobility and latency (FIG. 8C+D; F_{(1.21)}=9.53, p=0.0056; F_{(1.21)}=9.53, p=0.006, respectively) in the forced swim test. An interaction between treatment with OEA and ethanol intoxication in the immobility and latency parameters (F_{(1, 21)}=4.43, p=0.0475; F_{(1, 21)}=9.53, p=0.006, respectively) was also observed. Pre-treatment with OEA was able to prevent alcohol-induced depressive-like and anxiety symptoms, normalizing the swimming, immobility, and latency measurements (FIGS. 8 A, C and D; F_{(3, 21)}=2.935, p=0.05; F_{(3, 21)}=5.77, p=0.048; F_{(3, 21)}=4.17, p=0.018, respectively).

With respect to anxiety-like behaviors, the ANOVA Kruskal-Wallis test indicated that there was a general effect of the treatments on the percentage of entries (FIG. 8E; H=8.074, p=0.05) and on the time (FIG. 8F; H=7.702, p=0.0526) of permanence in the open arms of the elevated maze 24 h after ethanol intoxication. Although there was a clear trend that shows a clear anxiolytic effect of OEA in the animals treated with ethanol and in the control animals, Dunn's post-hoc test did not find significant effects in multiple comparisons. The effect of the treatments on the same anxiety measurements, the percentage of entries (FIG. 8G) and the time (FIG. 8H) of permanence in the open arms, was not significant 12 days after ethanol intoxication (H=6.377, p=0.09 and H=5.619, p=0.13, n.s., respectively).

### LITERATURE

Adachi J, Mizoi Y, Fukunaga T, Ogawa Y, Ueno Y & Imamichi, H (1991) Degrees of alcohol intoxication in 117 hospitalized cases. J Stud Alcohol 52: 448-453.
Agartz I, Momenan R, Rawlings R R, Kerich M J & Hommer D W (1999) Hippocampal volume in patients with alcohol dependence. Arch Gen Psychiatry 56: 356-363.
Alfonso-Loeches S, Pascual-Lucas M, Blanco A M, Sanchez-Vera I & Guerri C (2010) Pivotal role of TLR4 receptors in alcohol-induced neuroinflammation and brain damage. J Neurosci 30: 8285-8295.
Bilbao A, Blanco E, Luque-Rojas M J, Suarez J, Palomino A, Vida M, Araos P, Bermudez-Silva F J, Fernandez-Espejo E, Spanagel R & Rodriguez de Fonseca F (2013) Oleoylethanolamide dose-dependently attenuates cocaine-induced behaviours through a PPARalpha receptor-independent mechanism. Addict Biol 18: 78-87.
Bilbao A, Serrano A, Cippitelli A, Pavón F J, Giuffrida A, Suárez J, Garcia-Marchena N, Baixeiras E, Gómez de Heras R, Orio L, Alén F, Ciccocioppo R, Cravatt B F, Parsons L H, Piomelli D, Rodriguez de Fonseca F (2015) Role of the Satiety Factor Oleoylethanolamide in Alcoholism. Addiction Biology (in press).
Blednov Y A, Benavidez J M, Geil C, Perra S, Morikawa H & Harris R A (2011) Activation of inflammatory signaling by lipopolysaccharide produces a prolonged increase of voluntary alcohol intake in mice. Brain Behav Immun 25 Suppl 1: S92-S105.
Breese G R, Knapp D J, Overstreet D H, Navarro M, Wills T A & Angel R A (2008) Repeated lipopolysaccharide (LPS) or cytokine treatments sensitize ethanol withdrawal-induced anxiety-like behavior. Neuropsychopharmacology: official publication of the American College of Neuropsychopharmacology 33: 867-876.
Cippitelli A, Damadzic R, Hamelink C, Brunnquell M, Thorsell A, Heilig M & Eskay R L (2014) Binge-like ethanol consumption increases corticosterone levels and neurodegeneration whereas occupancy of type I I glucocorticoid receptors with mifepristone is neuroprotective. Addict Biol 19:27-36.
Collins M A, Tajuddin N, Moon K H, Kim H Y, Nixon K & Neafsey E. J (2014) Alcohol, phospholipase A2-associated neuroinflammation, and omega3 docosahexaenoic acid protection. Mol Neurobiol 50:239-245.
Collins M A, Zou J Y & Neafsey E J (1998) Brain damage due to episodic alcohol exposure in vivo and in vitro: furosemide neuroprotection implicates edema-based mechanism. FASEB journal: official publication of the Federation of American Societies for Experimental Biology 12:221-230.
Crews F, Nixon K, Kim D, Joseph J, Shukitt-Hale B, Qin L & Zou J (2006) BHT blocks N F-kappaB activation and ethanol-induced brain damage. Alcohol Clin Exp Res 30:1938-1949.
Crews F T, Braun C J, Hoplight B, Switzer R C, 3rd & Knapp D J (2000) Binge ethanol consumption causes differential brain damage in young adolescent rats compared with adult rats. Alcohol Clin Exp Res 24:1712-1723.
Crews F T, Collins M A, Dlugos C, Littleton J, Wilkins L, Neafsey E J, Pentney R, Snell L D, Tabakoff B, Zou J & Noronha A (2004) Alcohol-induced neurodegeneration: when, where and why? Alcohol Clin Exp Res 28: 350-364.
Crews, F. T., Qin, L., Sheedy, D., Vetreno, R. P. & Zou, J. (2013) High mobility group box 1/Toll-like receptor danger signaling increases brain neuroimmune activation in alcohol dependence. Biol Psychiatry 73:602-612.
Dantzer R, O'Connor J C, Freund G G, Johnson R W, Kelley K W (2008) From inflammation to sickness and depression: when the immune system subjugates the brain. Nat Rev Neurosci 9(1):46-56.
Fernandez-Lizarbe, S., Montesinos, J. & Guerri, C. (2013) Ethanol induces TLR4/TLR2 association, triggering an inflammatory response in microglial cells. J Neurochem 126: 261-273.
Fu J, Gaetani S, Oveisi F, Lo Verme J, Serrano A, Rodriguez De Fonseca F, Rosengarth A, Luecke H, Di Giacomo B, Tarzia G & Piomelli D (2003) Oleylethanolamide regulates feeding and body weight through activation of the nuclear receptor PPAR-alpha. Nature 425: 90-93.
Gonzalez-Aparicio R, Blanco E, Serrano A, Pavon F J, Parsons L H, Maldonado R, Robledo P, Fernandez-Espejo E & de Fonseca F R (2014) The systemic administration of oleoylethanolamide exerts neuroprotection of the nigrostriatal system in experimental Parkinsonism. Int J Neuropsychopharmacol 17: 455-468.
Hamelink C, Hampson A, Wink D A, Eiden L E & Eskay R L (2005) Comparison of cannabidiol, antioxidants, and diuretics in reversing binge ethanol-induced neurotoxicity. J Pharmacol Exp Ther 314: 780-788.
Haorah J, Ramirez S H, Floreani N, Gorantla S, Morsey B & Persidsky Y (2008) Mechanism of alcohol-induced oxidative stress and neuronal injury. Free radical biology & medicine 45: 1542-1550.
He J & Crews F T (2008) Increased MCP-1 and microglia in various regions of the human alcoholic brain. Exp Neurol 210: 349-358.
Hunt W A (1993) Are binge drinkers more at risk of developing brain damage? Alcohol 10: 559-561.
Jin P, Yu H L, Tian L, Zhang F & Quan Z S (2015) Antidepressant-like effects of oleoylethanolamide in a mouse model of chronic unpredictable mild stress. Pharmacol Biochem Behav 133: 146-154.
Knapp D J & Crews F T (1999) Induction of cyclooxygenase-2 in brain during acute and chronic ethanol treatment and ethanol withdrawal. Alcohol Clin Exp Res 23: 633-643.
Leclercq S, Cani P D, Neyrinck A M, Starkel P, Jamar F, Mikolajczak M, Delzenne N M & de Timary P (2012) Role of intestinal permeability and inflammation in the biological and behavioral control of alcohol-dependent subjects. Brain Behav Immun 26: 911-918.
Lemmers B, Salmena L, Bidère N, Su H, Matysiak-Zablocki E, Murakami K, Ohashi P S, Jurisicova A, Lenardo M, Hakem R, Hakem A. Essential role for caspase-8 in toll-like receptors and NfκB signaling. The J of Biol Chem 282:7416-7423.
Liu W, Kato M, Akhand A A, Hayakawa A, Suzuki H, Miyata T, Kurokawa K, Hotta Y, Ishikawa N, Nakashima I. 4-hydroxynonenal induces a cellular redox status-related activation of the caspase cascade for apoptotic cell death. J Cell Sci 113: 635:641.
Liu J W, Zhu H T, Chen K L, Qiu C, Tang K F, Niu X L (2013) Selenium attenuates high glucose-induced ROS/TLR4 involved apoptosis of rat cardiomyocyte. Bio Trace Elem Res 156:262-270
Majchrowicz E (1975) Induction of physical dependence upon ethanol and the associated behavioral changes in rats. Psychopharmacologia 43: 245-254.
McClain C J & Cohen D A (1989) Increased tumor necrosis factor production by monocytes in alcoholic hepatitis. Hepatology 9: 349-351.
McClain C J, Song Z, Barve S S, Hill D B & Deaciuc I (2004) Recent advances in alcoholic liver disease. I V. Dysregulated cytokine metabolism in alcoholic liver disease. Am J Physiol Gastrointest Liver Physiol 287:G497-502.
Melis M, Pillolla G, Luchicchi A, Muntoni A L, Yasar S, Goldberg S R & Pistis M. (2008) Endogenous fatty acid ethanolamides suppress nicotine-induced activation of mesolimbic dopamine neurons through nuclear receptors. J Neurosci 28: 13985-13994.
Montesinos, J., Pascual, M., Pla, A., Maldonado, C., Rodriguez-Arias, M., Minarro, J. & Guerri, C. (2015) TLR4 elimination prevents synaptic and myelin alterations and long-term cognitive dysfunctions in adolescent mice with intermittent ethanol treatment. Brain Behav Immun 45: 233-244.
Obernier J A, Bouldin T W & Crews F T (2002a) Binge ethanol exposure in adult rats causes necrotic cell death. Alcohol Clin Exp Res 26: 547-557.
Obernier J A, White A M, Swartzwelder H S & Crews F T (2002b) Cognitive deficits and CNS damage after a 4-day binge ethanol exposure in rats. Pharmacol Biochem Behav 72: 521-532.
Pascual, M., Balino, P., Alfonso-Loeches, S., Aragon, C. M. & Guerri, C. (2011) Impact of TLR4 on behavioral and cognitive dysfunctions associated with alcohol-induced neuroinflammatory damage. Brain Behav Immun 25 Suppl 1: S80-91.
Pascual, M., Balino, P., Aragon, C. M. & Guerri, C. (2015) Cytokines and chemokines as biomarkers of ethanol-induced neuroinflammation and anxiety-related behavior: role of TLR4 and TLR2. Neuropharmacology 89: 352-359.
Pascual, M., Pla, A., Minarro, J. & Guerri, C. (2014) Neuroimmune activation and myelin changes in adolescent rats exposed to high-dose alcohol and associated cognitive dysfunction: a review with reference to human adolescent drinking. Alcohol Alcohol 49: 187-192.
Plaza-Zabala A, Berrendero F, Suarez J, Bermudez-Silva F J, Fernandez-Espejo E, Serrano A, Pavon F J, Parsons L H, De Fonseca F R, Maldonado R, Robledo P. (2010) Effects of the endogenous PPAR-alpha agonist, oleoylethanolamide on MDMA-induced cognitive deficits in mice. Synapse 64(5):379-89.
Qin L & Crews F T (2012) NADPH oxidase and reactive oxygen species contribute to alcohol-induced microglial activation and neurodegeneration. J Neuroinflammation 9:5.
Qin L, He J, Hanes R N, Pluzarev O, Hong J S, Crews F T (2008) Increased systemic and brain cytokine production and neuroinflammation by endotoxin following ethanol treatment. J Neuroinflammation. 5:10.
Ribeiro A, Pontis S, Mengatto L, Armirotti A, Chiurchiu V, Capurro V, Fiasella A, Nuzzi A, Romeo, E, Moreno-Sanz G, Maccarrone M, Reggiani A, Tarzia G, Mor M, Bertozzi F, Bandiera T & Piomelli D (2015) A Potent Systemically Active N-Acylethanolamine Acid Amidase Inhibitor that Suppresses Inflammation and Human Macrophage Activation. ACS Chem Biol.
Rodriguez de Fonseca F, Navarro M, Gomez R, Escuredo L, Nava F, Fu J, Murillo-Rodriguez E, Giuffrida A, LoVerme J, Gaetani S, Kathuria S, Gall C & Piomelli D. (2001) An anorexic lipid mediator regulated by feeding. Nature 414: 209-212.
Sayd A, Anton M, Alen F, Caso J R, Pavon J, Leza J C, Rodriguez de Fonseca F, Garcia-Bueno B & Orio L (2014) Systemic administration of oleoylethanolamide protects from neuroinflammation and anhedonia induced by LPS in rats. Int J Neuropsychopharmacol: 18.
Smaga I, Bystrowska B, Gawkinski D, Pomierny B, Stankowicz P, Filip M (2014) Antidepressants and changes in concentration of endocannabinoids and N-acylethanolamides in rat brain structures. Neurotox Res 26:190-206.
Snyder H, Williams D, Zink B. & Reilly K (1992) Accuracy of blood ethanol determination using serum osmolality. J Emerg Med 10: 129-133.
Stahel P, Smith W R, Bruchis J, Rabb C H (2008) Peroxisome proliferator-activated receptors: “key” regulators of neuroinflammation after traumatic brain injury. PPAR Res 2008: 538141.
Sun Y, Alexander S P, Garle M J, Gibson C L, Hewitt K, Murphy S P, Kendall D A & Bennett A J (2007) Cannabinoid activation of PPAR alpha; a novel neuroprotective mechanism. Br J Pharmacol 152: 734-743.
Tajuddin N, Moon K H, Marshall S A, Nixon K, Neafsey E J, Kim H Y & Collins M A (2014) Neuroinflammation and neurodegeneration in adult rat brain from binge ethanol exposure: abrogation by docosahexaenoic acid. PLoS One 9:e101223.
Tiwari M, Kotwal J, Kotwal A, Mishra P, Dutta V & Chopra S (2013) Correlation of haemoglobin and red cell indices with serum ferritin in Indian women in second and third trimester of pregnancy. Medical journal, Armed Forces India 69: 31-36.
Tiwari V & Chopra K (2012) Attenuation of oxidative stress, neuroinflammation, and apoptosis by curcumin prevents cognitive deficits in rats postnatally exposed to ethanol. Psychopharmacology (Berl) 224: 519-535.
Tiwari V & Chopra K (2013) Resveratrol abrogates alcohol-induced cognitive deficits by attenuating oxidative-nitrosative stress and inflammatory cascade in the adult rat brain. Neurochemistry international 62:861-869.
Valles S L, Blanco A M, Pascual M & Guerri C (2004) Chronic ethanol treatment enhances inflammatory mediators and cell death in the brain and in astrocytes. Brain Pathol 14:365-371.
Vetreno R P & Crews F T (2012) Adolescent binge drinking increases expression of the danger signal receptor agonist HMGB1 and Toll-like receptors in the adult prefrontal cortex. Neuroscience 226: 475-488.
Viceconte N, Burguillos M A, Herrera A J, De Pablos R M, Joseph B, Venero J L (2015) Neuromelanin activates proinflammatory microglia through a caspase-8-dependent mechanism. J Neuroinflammation 12:5.
Yang, G., Meng, Y., Li, W., Yong, Y., Fan, Z., Ding, H., Wei, Y., Luo, J. & Ke, Z. J. (2011) Neuronal MCP-1 mediates microglia recruitment and neurodegeneration induced by the mild impairment of oxidative metabolism. Brain Pathol 21: 279-297.
Zahr, N. M., Alt, C., Mayer, D., Rohlfing, T., Manning-Bog, A., Luong, R., Sullivan, E. V. & Pfefferbaum, A. (2014) Associations between in vivo neuroimaging and postmortem brain cytokine markers in a rodent model of Wernicke's encephalopathy. Exp Neurol 261: 109-119.
Zou, J. Y. & Crews, F. T. (2014) Release of neuronal HMGB1 by ethanol through decreased HDAC activity activates brain neuroimmune signaling. PLoS One 9: e87915.

1. A method for the prevention, relief or therapeutic treatment of alcohol consumption disorders, comprising administering a composition comprising a compound of general formula (I): 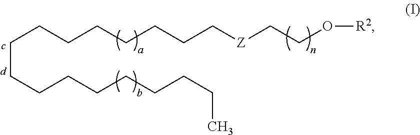
or a pharmaceutically acceptable salt, ester, tautomer, solvates or hydrates thereof, to a subject in need thereof,
wherein:
n is an integer from 0 and to 5;
a and b are determined by the following formula: 0≤(a+b)≤4;
Z is a group selected from the group consisting of —C(O)N(R⁰)—, —(R⁰)NC(O)—, —(O)CO, O, NR⁰ and S;
R⁰ and R² are independently selected from the group consisting of a substituted or unsubstituted alkyl, hydrogen, a substituted or unsubstituted (C1-C6) alkyl, a substituted or unsubstituted (C1-C6) acyl, homoalkyl, and aryl, and wherein up to eight hydrogen atoms of the compound is substituted by methyl or a double bond; and
the bond between c and d is saturated or unsaturated.

2. The method of claim 1, wherein the compound has the formula (II), 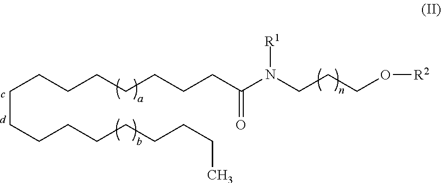
or a pharmaceutically acceptable salt, ester, tautomer, solvate or hydrate thereof,
wherein:
n is an integer from 0 to 4;
a and b are determined by the following formula: 0<(a+b)≤3;
R¹ and R² are independently selected from the group consisting of hydrogen (H), a substituted or unsubstituted (C1-C6) alkyl, and a substituted or unsubstituted (C1-C6) acyl, and wherein up to eight hydrogen atoms of the compound are substituted by methyl or a double bond; and
the bond between c and d is saturated or unsaturated.

3. The method of claim 1, wherein a=1 and b=1.

4. The method of claim 1, wherein n=0 or 1.

5. The method of claim 2, wherein R¹ and R² are each hydrogen (H).

6. The method of claim 1, wherein the bond between carbon c and carbon d is a double bond.

7. The method of claim 1, wherein the compound of formula (I) is an acylethanolamide.

8. The method of claim 7, wherein the acylethanolamide is selected from the group consisting of oleoylethanolamide (OEA), palmitoylethanolamide (PEA), stearoylethanolamide (SEA) and combinations thereof.

9. The method of claim 8, wherein the acylethanolamide is oleoylethanolamide (OEA) of formula (III)

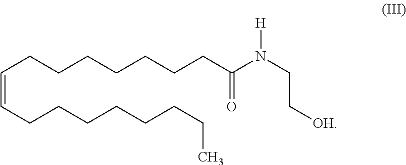 [SMILES: CCCCCCCC/C=C\CCCCCCCC(=O)NCCO]

10. The method of claim 1, wherein the alcohol consumption disorders are selected from the group consisting of: alcohol intoxication, pathological inebriation, and alcohol dependence syndrome.

11. The method of claim 10, wherein the alcohol consumption disorder is alcohol intoxication or pathological inebriation.

12. The method of claim 1, wherein the alcohol consumption disorder is alcohol dependence syndrome.

13. The method of claim 1, wherein the method is for prevention of alcohol consumption disorders, and the administration of the compound is performed prior to intake of alcohol.

14. The method of claim 1, wherein the method is for prevention of alcohol consumption disorders, and the administration of the compound is performed during intake of alcohol.

15. The method of claim 1, wherein the composition further comprises a second active ingredient selected from the group consisting of ibuprofen, paracetamol, vitamin B12, caffeine and combinations thereof.

16. The method of claim 1, wherein the composition further comprises electrolytes, an isotonic beverage, natural juices, thistle teas, caffeine or a combination thereof.

17. A nutraceutical composition comprising a compound of claim 1 and a pharmaceutically acceptable diluent or carrier.

18. The nutraceutical composition of claim 17, wherein said composition further comprises electrolytes, an isotonic beverage, natural juices, thistle teas, caffeine or a combination thereof.
